(19) United States
(12) Patent Application Publication
(10) Pub. No.: US 2012/0258451 A1
(43) Pub. Date: Oct. 11, 2012

(54) LASER ISOLATION OF VIABLE CELLS

(75) Inventor: Irina Vitaly KLIMANSKAYA, Upton, MA (US)

(73) Assignee: Advanced Cell Technology, Inc., Marlborough, MA (US)

(21) Appl. No.: 13/441,617

(22) Filed: Apr. 6, 2012

Related U.S. Application Data
(60) Provisional application No. 61/473,422, filed on Apr. 8, 2011.

(51) Int. Cl. C12N 13/00; C12Q 1/02; C12Q 1/68; G01N 33/53

(52) U.S. Cl. 435/6.1; 435/173.9; 435/7.1; 435/29

(57) ABSTRACT

Methods for laser microdissection isolation of viable cells are provided. Cells of a desired type may be isolated from a diverse population, optionally with detection and exclusion of undesired cells. Desired cells may be isolated from a population that arose from differentiation of pluripotent cells, preferably embryonic stem cells or induced pluripotent stem cells, and undifferentiated stem cells may be detected and excluded from selection including the isolation of RPE cells sleeted based on morphology (e.g., characteristic mottled appearance) from a population of ES cells. The cells isolated by these methods, including RPE cells, may be essentially free of undifferentiated cells and thus suitable for use in cell-based therapies.

### BACKGROUND

1. Field of the Invention

The invention relates to laser microdissection methods for obtaining viable cells. The invention provides methods for the isolation of viable cells differentiated from pluripotent or multipotent cells, preferably embryonic stem cells or induced pluripotent stem cells (iPSCs), including ocular cells such as retinal pigment epithelium cells, iris pigment epithelium cells, vision-associated neural cells, lens cells, rods, cones, or corneal cells. The methods provided by the invention may provide high-purity cell cultures suitable for cell-based therapies.

2. Description of the Related Art

Laser microdissection methods may allow for the isolation of an individual cell to be separated from the surrounding preparation (e.g., a tissue section) by the laser beam, and then released. The released cells may then be moved to a collection device, for example by mechanical means or a laser-induced transport process with the aid of a laser pulse. See Thalhammer, et al. (2003) Laser Methods in Medicine and Biology 13(5): 681-691 and Murray & Curran (2005) Laser Microdissection: Methods and Protocols 293 from Methods in Molecular Biology.

Laser-mediated micromanipulation (LMM), a laser microdissection technique, uses a fine, focused laser beam to sever the connections between desired cells and the surrounding portion of the specimen. The desired cells may then be removed by physical manipulation or by “catapulting” (e.g., a laser pulse imparts momentum to the desired piece and allows to be moved without being touched). See Thalhammer, et al. (2003) Laser Methods in Medicine and Biology 13(5): 681-691.

LMM has been used for molecular analysis of individual cells or groups of cells isolated from tissue samples. For example, after ethanol fixation, individual aveolar macrophages were isolated by laser photolysis of undesired adjacent cells followed by mechanical picking with a needle, after which individual macrophages were transferred into a reaction tube for RNA extraction and RT-PCR analysis (Fink, et al. (1998) Nature Medicine 4: 1329-1333). Additional exemplary LMM methods are described in U.S. Pat. No. 5,998,129; U.S. Patent Application Publication No. 2009/0002682; Vogel, et al. (2007) Methods Cell Biol. 82: 153-205; Stich, et al. (2003) Pathol Res Pract. 199(6): 405-9; and Mayer, et al. (2002) Methods Enzymol. 356: 25-33.

In an another laser microdissection technique, laser capture microdissection (LCM), a thermoplastic film is placed over the sample and caused to selectively adhere to the desired cells by a laser pulse that heats part of the thermoplastic film and causes it to adhere to the cells. The cells are then removed with the film. Cells isolated by LCM have been used for analysis of DNA, RNA, and protein. See, e.g., Buck, et al. (1996) Science 274(5289): 998-1001, Bonner, et al. (1997) Science 278(5342): 1481-1483; U.S. Pat. No. 6,184,973; U.S. Pat. No. 6,897,038; U.S. Pat. No. 5,859,699; U.S. Pat. No. 6,495,195; U.S. Pat. No. 6,100,051; U.S. Pat. No. 6,720,191; U.S. Pat. No. 6,700,653; and U.S. Pat. No. 6,743,601.

The LCM technique has been used to isolate cells for extraction and analysis of their contents. For example, ethanol-fixed cells have been isolated by LCM from post-mortem human eyes for RT-PCR measurement of alterations in gene expression in retinal pigment epithelium cells adjacent to basal deposits. Yamada, et al. (2006) Exp Eye Res. 82(5): 840-8. Similar techniques—again using post-mortem human eyes, ethanol-fixation, and RT-PCR analysis—have been used to identify differences in gene expression between retinal pigment epithelium cells isolated by LCM from the different regions of the eye. Ishibashi, et al. (2004) Invest Ophthalmol Vis Sci. 45(9): 3291-301. Retinal pigment epithelium and other cells have also been isolated by LCM from frozen mouse eye sections for RT-PCR to determine which specific cell type(s) expressed cytokines in inflamed eyes. Foxman, et al. (2002) J Immunol. 168(5): 2483-92. These references report using LCM to isolate non-viable cells for molecular analysis but do not report using LCM to isolate viable cells.

Laser microdissection and pressure catapulting (LMPC), a laser microdissection technique, involves placing a biological sample directly on top of a thermoplastic polyethyelene napthalate membrane that covers the glass slide. The membrane acts as a support (scaffolding) to allow for catapulting relatively large amounts of intact material at a time. A focused laser beam is used to cut out an area of the membrane and corresponding biological sample, and the beam is then defocused and the energy used to catapult the membrane and material from the slide. The catapulted sample may be captured in an aqueous media positioned directly above the cut area. See Kuhn, et al. (2007) Am J Phyiol. Heart Circ. Physiol. 292: H1245-H1253, H1245. This method has been used to isolate embryonic-stem cells derived cardiomyocytes. Khuram, et al. (2006) Toxicological Sciences 90(1): 149-158, abstract.

However, there remains a need for improved techniques for isolating ocular cells (e.g., retinal pigment epithelium cells, iris pigment epithelium cells, vision-associated neural cells, lens cells, rods, cones, and corneal cells) that remain viable and which are of sufficient purity as to be useful for cell-based therapies.

### Retinal Pigment Epithelium (RPE)

The retinal pigment epithelium (RPE) is the pigmented cell layer outside the neurosensory retina between the underlying choroid (the layer of blood vessels behind the retina) and overlying retinal visual cells (e.g., photoreceptors—rods and cones). The RPE is critical to the function and health of photoreceptors and the retina. The RPE maintains photoreceptor function by recycling photopigments, delivering, metabolizing, and storing vitamin A, phagocytosing rod photoreceptor outer segments, transporting iron and small molecules between the retina and choroid, maintaining Bruch's membrane and absorbing stray light to allow better image resolution. Engelmann & Valtink (2004) Clinical and Experimental Ophthalmology 242(1): 65-67; See also Irina Klimanskaya (2009) Retinal Pigment Epithelium Derived From Embryonic Stem Cells, in STEM CELL ANTHOLOGY 335-346.

Mature RPE is characterized by its cobblestone cellular morphology of black pigmented cells and RPE cell markers including cellular retinaldehyde-binding protein (CRALBP), a 36-kD cytoplasmic retinaldehyde-binding protein that is also found in apical microvilli (Eisenfeld, et al. (1985) Experimental Research 41(3): 299-304); RPE65, a 65 kD cytoplasmic protein involved in retinoid metabolism (Ma, et al. (2001) Invest Opthalmol Vis Sci. 42(7): 1429-35; Redmond (2009) Exp Eye Res. 88(5): 846-847); bestrophin, a membrane localized 68 kD product of the Best vitelliform macular dystrophy gene (VMD2) (Marmorstein, et al. (2000) PNAS 97(23): 12758-12763), and pigment epithelium derived factor (PEDF), a 48-kD secreted protein with angiostatic properties (Karakousis, et al. (2001) Molecular Vision 7: 154-163; Jablonski, et al. (2000) The Journal of Neuroscience 20(19): 7149-7157).

Degeneration of the RPE may cause retinal detachment, retinal dysplasia, or retinal atrophy that is associated with a number of vision-altering ailments that result in photoreceptor damage and blindness, such as, choroideremia, diabetic retinopathy, macular degeneration (including age-related macular degeneration), retinitis pigmentosa, and Stargardt's Disease (fundus flavimaculatus). See WO 2009/051671.

### RPE Cells in Medicine

Given the importance of the RPE in maintaining visual and retinal health, the RPE and methodologies for producing RPE cells in vitro are of considerable interest. See Lund, et al. (2001) Progress in Retinal and Eye Research 20(4): 415-449. For example, a study reported in Gouras, et al. (2002) Investigative Ophthalmology & Visual Science 43(10): 3307-311 describes the transplantation of RPE cells from normal mice into transgenic RPE65^{-/-} mice (a mouse model of retinal degeneration). Gouras discloses that the transplantation of healthy RPE cells slowed the retinal degeneration in the RPE65^{-/-} mice but after 3.7 weeks, its salubrious effect began to diminish. Treumer, et al. (2007) Br J Opthalmol 91: 349-353 describes the successfully transplantation of autologous RPE-choroid sheet after removal of a subfoveal choroidal neovascularization (CNV) in patients with age related macular degeneration (AMD), but this procedure only resulted in a moderate increase in mean visual acuity.

However, RPE cells sourced from human donors has several intractable problems. First, is the shortage of eye donors, and the current need is beyond what could be met by donated eye tissue. For example, RPE cells sourced from human donors are an inherently limited pool of available tissue that prevent it from scaling up for widespread use. Second, the RPE cells from human donors may be contaminated with pathogens and may have genetic defects. Third, donated RPE cells are derived from cadavers. The cadaver-sourced RPE cells have an additional problem of age where the RPE cells are may be close to senesce (e.g., shorter telomeres) and thus have a limited useful lifespan following transplantation. Reliance on RPE cells derived from fetal tissue does not solve this problem because these cells have shown a very low proliferative potential. Further, fetal cells vary widely from batch to batch and must be characterized for safety before transplantation. See, e.g., Irina Klimanskaya (2009) Retinal Pigment Epithelium Derived From Embryonic Stem Cells, in STEM CELL ANTHOLOGY 335-346. Any human sourced tissue may also have problems with tissue compatibility leading to immunological response (graft-rejection). Also, cadaver-sourced RPE cells may not be of sufficient quality as to be useful in transplantation (e.g., the cells may not be stable or functional). Fourth, sourcing RPE cells from human donors may incur donor consent problems and must pass regulatory obstacles, complicating the harvesting and use of RPE cells for therapy. Fifth, a fundamental limitation is that the RPE cells transplanted in an autologous transplantation carry the same genetic information that may have lead to the development of AMD. See, e.g., Binder, et al. (2007) Progress in Retinal and Eye Research 26(5): 516-554. Sixth, the RPE cells used in autologous transplantation are already cells that are close to senesce, as AMD may develop in older patients. Thus, a shorter useful lifespan of the RPE cells limits their utility in therapeutic applications (e.g., the RPE cells may not transplant well and are less likely to last long enough for more complete recovery of vision). Seventh, to be successful in long-term therapies, the transplanted RPE cells must integrate into the RPE layer and communicate with the choroid and photoreceptors. Eighth, in AMD patients and elderly patients also suffer from degeneration of the Bruch's membrane, complicating RPE cell transplantation. See Gullapalli, et al. (2005) Exp Eye Res. 80(2): 235-48. Thus there exists a great need for a source of RPE cells for therapeutic uses and human embryonic stem cells (hES) are considered a promising source of replacement RPE cells for clinical use. See Idelson, et al. (2009) Cell Stem Cell 5: 396-408.

Methods for the systematic directed matter for the production of large numbers of RPE cells have been described (e.g., PCT/US2010/57056 and WO 2009/051671). For example, when differentiated cells are to be produced from ES cells for transplantation, there is concern that presence of a few residual ES cells could give rise to a tumor or teratoma. Some assurance of safety can come from administering the cell preparation to an animal (e.g., an immune compromised animal). However, animal testing alone may be considered insufficient because a human ES cell may be more prone to produce a teratoma in a human host than in the animal model.

Additionally, methods for producing RPE cells by differentiation of RPE cells from pluripotent stem cells produces a heterogeneous population of cells comprising RPE cells and other differentiated cells (e.g., neural rosettes). The standard method of manual selection relies on the operator's skill and experience in selecting the RPE cells and not the other differentiated cells. Moreover, manual selection of pigmented clusters is very tedious and fully relies on the operator's skills and judgment which may get impaired after several hours of such scrupulous selection and the microscope involving eye and back-straining work. Thus, it is desirable to provide methods that may decrease or eliminate the possibility of undesired residual undifferentiated ES cells in a cell population isolated from differentiated ES cells. Thus, there exists a need for a rapid method for the isolation of large numbers of RPE cells with sufficient purity as to be suitable for use in transplantation therapies.

### BRIEF SUMMARY OF THE INVENTION

In one aspect, the invention provides a method for isolating a viable cell morphologically distinguishable from other cells contained within a heterogeneous population of cells comprising (a) providing a planar carrier containing a heterogeneous cell population, (b) placing said planar carrier in a microscope coupled to a laser microdissection system, (c) selecting said desired cell, (d) excising said cell, and (e) collecting said cell.

In one embodiment, the population of cells may comprise human or primate cells. In another embodiment, the population may comprises both differentiated and undifferentiated cells. The undifferentiated cells may comprise embryonic stem cells (ESCs). The embryonic stem cells may be identified by detection of a detectable characteristic selected from the group consisting of presence in a round colony with clear margins; a high nucleus/cytoplasm ratio with prominent nucleoli; rounded cells that lie tightly packed with each other; and expression of at least one ES cell markers selected from the group consisting of OCT-4, Nanog, TRA-1-60, SSEA-3, SSEA-4, TRA-1-81, SOX2, and alkaline phosphatase.

In one embodiment, the cell population may be produced by differentiation of embryonic stem cells. In another embodiment, the differentiation of embryonic stem cells may comprise (a) allowing hES cell cultures to overgrow on MEF and form a thick multilayer of cells, or forming an embryoid body (EB) from hES cells; (b) culturing the hES cells multilayer of cells or EB for a sufficient time for the appearance of pigmented cells comprising brown pigment dispersed in their cytoplasm.

In one embodiment, the cell may be produced by culturing pigmented epithelial cells obtained from differentiated embryonic stem cells. In a further embodiment, the method may further comprise contacting said cell of step (a) with a vital stain. In another embodiment, the excising of step (d) may comprise removing the selected cells from the planar carrier using micromanipulation or laser catapulting. In a further embodiment, the collection of step (e) may comprise manual colony picking, micromanipulation, or laser capture.

In one aspect, the invention provides a method for isolating a viable differentiated cell which is morphologically distinguishable from other undifferentiated cells which both are contained a population of cells comprising (a) providing a planar carrier on which said population of cells containing said at least one differentiated cell is situated, (b) placing said planar carrier in a microscope coupled to a laser microdissection system, (c) selecting said differentiated cell, (d) excising said differentiated cell, (e) separating said differentiated cell from the planar carrier, and (f) collecting said differentiated cell.

In one embodiment, the population of cells may be a heterogeneous population. population comprises both differentiated and undifferentiated cells. In another embodiment, the undifferentiated cells comprise embryonic stem cells (ESCs). In another embodiment, the embryonic stem cells may be identified by detection of a detectable characteristic selected from the group consisting of presence in a round colony with clear margins; a high nucleus/cytoplasm ratio with prominent nucleoli; rounded cells that lie tightly packed with each other; and expression of at least one ES cell markers selected from the group consisting of OCT-4, Nanog, TRA-1-60, SSEA-3, SSEA-4, TRA-1-81, SOX2, and alkaline phosphatase.

In one embodiment, the cell population may be produced by differentiation of pluripotent stem cells. In another embodiment, the pluripotent cells may be selected from the group consisting of induced pluripotent stem (iPS) cells, embryonic stem (ES) cells, blastomeres, morula cells, embroid bodies, adult stem cells, hematopoietic stem cells, fetal stem cells, mesenchymal stem cells, postpartum stem cells, multipotent stem cells, and embryonic germ cells.

In one embodiment, the pluripotent stem cell may be an embryonic stem cell. In another embodiment, the differentiation of embryonic stem cells may comprise (a) allowing hES cell cultures to overgrow on MEF and form a thick multilayer of cells, or forming an embryoid body (EB) from hES cells; (b) culturing the hES cells multilayer of cells or EB for a sufficient time for the appearance of pigmented cells comprising brown pigment dispersed in their cytoplasm. In another embodiment, the differentiated cell may be produced by culturing pigmented epithelial cells obtained from differentiated embryonic stem cells.

In another embodiment, the method may further comprise contacting said cell of step (a) with a vital stain. In another embodiment, the excising of step (d) may comprise removing the selected cells from the planar carrier using micromanipulation or laser catapulting. In another embodiment, the collection of step (e) may comprise manual colony picking, micromanipulation, or laser capture.

In one aspect, the invention provides a method for isolating a viable cell from a heterogeneous population of cells comprising (a) providing a planar carrier on which said population of cells containing said at least one viable cell is situated, (b) placing said culture dish in a microscope coupled to a laser microdissection system, (c) selecting said viable cell, (d) excising said viable cell, (e) separating said viable cell from the planar carrier, and (f) collecting said viable cell.

In one embodiment, the population may comprise both differentiated and undifferentiated cells. In another embodiment, the undifferentiated cells may be pluripotent stem cells.

In one embodiment, the pluripotent stem cell may be an embryonic stem cell (ESC). In another embodiment, the embryonic stem cells may be identified by detection of a detectable characteristic selected from the group consisting of presence in a round colony with clear margins; a high nucleus/cytoplasm ratio with prominent nucleoli; rounded cells that lie tightly packed with each other; and expression of at least one ES cell markers selected from the group consisting of OCT-4, Nanog, TRA-1-60, SSEA-3, SSEA-4, TRA-1-81, SOX2, and alkaline phosphatase.

In one embodiment, the cell population is produced by differentiation of embryonic stem cells. In another embodiment, the differentiation of embryonic stem cells may comprise (a) allowing hES cell cultures to overgrow on MEF and form a thick multilayer of cells, or forming an embryoid body (EB) from hES cells; (b) culturing the hES cells multilayer of cells or EB for a sufficient time for the appearance of pigmented cells comprising brown pigment dispersed in their cytoplasm.

In one embodiment, the viable cell may be produced by culturing pigmented epithelial cells obtained from differentiated embryonic stem cells. In another embodiment, the method may further comprise contacting said cell of step (a) with a vital stain. In another embodiment, the excising of step (d) may comprise removing the selected cells from the planar carrier using micromanipulation or laser catapulting. In another embodiment, the collection of step (e) may comprise manual colony picking, micromanipulation, or laser capture.

In one aspect, the invention provides a method for isolating a RPE cell from a population of cells comprising (a) providing a planar carrier on which said population of cells is situated, (b) placing said planar carrier in a microscope coupled to a laser microdissection system, (c) selecting said at least one RPE cell, (d) excising said cell from undesired cells or other materials in target areas adjacent to the selected cells using laser light, thereby severing the connections between the selected cells and adjacent cells or other materials, and (e) collecting said RPE cell.

In one embodiment, the population of cells may be a heterogeneous population. In another embodiment, the population may comprise both differentiated and undifferentiated cells.

In one embodiment, the undifferentiated cells may comprise embryonic stem cells (ESCs). In another embodiment, the embryonic stem cells may be identified by detection of a detectable characteristic selected from the group consisting of presence in a round colony with clear margins; a high nucleus/cytoplasm ratio with prominent nucleoli; rounded cells that lie tightly packed with each other; and expression of at least one ES cell markers selected from the group consisting of OCT-4, Nanog, TRA-1-60, SSEA-3, SSEA-4, TRA-1-81, SOX2, and alkaline phosphatase.

In one embodiment, the cell population may be produced by differentiation of embryonic stem cells. In another embodiment, the differentiation of embryonic stem cells may comprise (a) allowing hES cell cultures to overgrow on MEF and form a thick multilayer of cells, or forming an embryoid body (EB) from hES cells; (b) culturing the hES cells multilayer of cells or EB for a sufficient time for the appearance of pigmented cells comprising brown pigment dispersed in their cytoplasm.

In one embodiment, the RPE cell is produced by culturing pigmented epithelial cells obtained from differentiated embryonic stem cells. In another embodiment, the method may further comprise contacting said cell of step (a) with a vital stain. In another embodiment, the excising of step (d) may comprise removing the selected cells from the planar carrier using micromanipulation or laser catapulting. In another embodiment, the collection of step (e) may comprise manual colony picking, micromanipulation, or laser capture.

In one aspect, the invention provides a method of isolating a viable RPE cell from a heterogeneous population of cells comprising (a) providing a planar carrier on which a cell population comprising at least one viable desired cell is situated; (b) selecting at least one desired cell to be isolated; (c) excising said at least one cell from undesired cells or other materials in target areas adjacent to the selected cells using laser light, thereby severing the connections between the selected cells and adjacent cells or other materials; and (d) separating the at least one selected cell from the planar carrier, thereby isolating the selected cells, wherein the isolated cells comprise viable desired cells, wherein said desired cells are of a desired cell type selected from the group consisting of iris pigment epithelium cells, vision-associated neural cells, lens cells, rod cells, cone cells, or corneal cells.

In one embodiment, the heterogeneous population may comprise both differentiated and undifferentiated cells. In another embodiment, the undifferentiated cells may comprise embryonic stem cells (ESCs). In another embodiment, the embryonic stem cells may be identified by detection of a detectable characteristic selected from the group consisting of presence in a round colony with clear margins; a high nucleus/cytoplasm ratio with prominent nucleoli; rounded cells that lie tightly packed with each other; and expression of at least one ES cell markers selected from the group consisting of OCT-4, Nanog, TRA-1-60, SSEA-3, SSEA-4, TRA-1-81, SOX2, and alkaline phosphatase.

In one embodiment, the heterogeneous cell population may be produced by differentiation of embryonic stem cells. In another embodiment, the differentiation of embryonic stem cells may comprise (a) allowing hES cell cultures to overgrow on MEF and form a thick multilayer of cells, or forming an embryoid body (EB) from hES cells; (b) culturing the hES cells multilayer of cells or EB for a sufficient time for the appearance of pigmented cells comprising brown pigment dispersed in their cytoplasm.

In one embodiment, the RPE cell may be produced by culturing pigmented epithelial cells obtained from differentiated embryonic stem cells. In another embodiment, the method may further comprise contacting said cell of step (a) with a vital stain. In another embodiment, the excising of step (d) may comprise removing the selected cells from the planar carrier using micromanipulation or laser catapulting. In another embodiment, the collection of step (e) may comprise manual colony picking, micromanipulation, or laser capture.

In one embodiment, the viable cell may be a RPE cell selected based on pigmentation. In another embodiment, the viable cell may be an RPE cell selected based on at least one detectable characteristic of RPE cells. The detectable characteristic of RPE cells may be at least one of presence of brown pigmentation accumulated within the cytoplasm, a cobblestone, epithelial-like morphology, or expression of at least one RPE cell markers. The RPE cell marker may be selected from the group consisting of bestrophin, RPE65, CRALBP, and PEDF. The RPE marker may be detected by a method selected from the group consisting of binding to an antibody directly or indirectly coupled to a detectable label; incubation with magnetic beads—conjugated antibodies; detecting the expression of a fluorescent protein; detecting an intracellular mRNA, detecting an intracellular protein; and detecting an intracellular small molecule. The viable cell may exhibit at least one detectable characteristics of RPE cells. The detectable characteristics of RPE cells may be morphology or expression of at least one RPE cell markers. The RPE cell marker may be selected from the group consisting of markers identified in Table 1.

In one embodiment, the differentiated cell may be a RPE cell selected based on pigmentation. In another embodiment, the differentiated cell may be an RPE cell selected based on at least one detectable characteristic of RPE cells. The detectable characteristic of RPE cells may be at least one of presence of brown pigmentation accumulated within the cytoplasm, a cobblestone, epithelial-like morphology, or expression of at least one RPE cell markers. The RPE cell marker may be selected from the group consisting of bestrophin, RPE65, CRALBP, and PEDF. The RPE marker may be detected by a method selected from the group consisting of binding to an antibody directly or indirectly coupled to a detectable label; incubation with magnetic beads-conjugated antibodies; detecting the expression of a fluorescent protein; detecting an intracellular mRNA, detecting an intracellular protein; and detecting an intracellular small molecule. The differentiated cell may exhibit at least one detectable characteristics of RPE cells. The detectable characteristics of RPE cells may be morphology or expression of at least one RPE cell markers. The RPE cell marker may be selected from the group consisting of markers identified in Table 1.

In one embodiment, the viable cell may be differentiated from one or more pluripotent cells. In another embodiment, the pluripotent cells may be selected from the group consisting of induced pluripotent stem (iPS) cells, embryonic stem (ES) cells, blastomeres, morula cells, embroid bodies, adult stem cells, hematopoietic stem cells, fetal stem cells, mesenchymal stem cells, postpartum stem cells, multipotent stem cells, and embryonic germ cells. In a further embodiment, the pluripotent stem cell may be an embryonic stem cell. In a still further embodiment, the pluripotent stem cell may be a human embryonic stem cell.

In one embodiment, the differentiated cell may be differentiated from one or more pluripotent cells. In another embodiment, the pluripotent cells may be selected from the group consisting of induced pluripotent stem (iPS) cells, embryonic stem (ES) cells, blastomeres, morula cells, embroid bodies, adult stem cells, hematopoietic stem cells, fetal stem cells, mesenchymal stem cells, postpartum stem cells, multipotent stem cells, and embryonic germ cells. In a further embodiment, the pluripotent stem cell may be an embryonic stem cell. In a still further embodiment, the pluripotent stem cell may be a human embryonic stem cell.

In one embodiment, the viable cell may be a differentiated cell. In one embodiment, the differentiated cell may be a RPE cell. In a further embodiment, the viable cell may be a RPE cell. In another embodiment, the RPE cell may be a retinal pigment epithelium (RPE) cell. In another embodiment, the RPE cell may be selected from the group consisting of iris pigment epithelium cells, vision-associated neural cells, lens cells, rod cells, cone cells, or corneal cells. In another embodiment, the differentiated cell may be selected from the group consisting of iris pigment epithelium cells, vision-associated neural cells, lens cells, rod cells, cone cells, or corneal cells. In another embodiment, the viable cell may be selected from the group consisting of iris pigment epithelium cells, vision-associated neural cells, lens cells, rod cells, cone cells, or corneal cells.

In a one embodiment, the viable cell may be a human viable cell. In another embodiment, the viable cell may be a non-human animal, non-human primate, murine, ovine, bovine, canine, porcine, chimpanzee, cynomolgus monkey, baboon, Old World monkey, caprine, equine, ungulate, or feline viable cell. In a one embodiment, the differentiated cell may be a human viable cell. In another embodiment, the differentiated cell may be a non-human animal, non-human primate, murine, ovine, bovine, canine, porcine, chimpanzee, cynomolgus monkey, baboon, Old World monkey, caprine, equine, ungulate, or feline differentiated cell. In a one embodiment, the RPE cell may be a human viable cell. In another embodiment, the viable cell may be a non-human animal, non-human primate, murine, ovine, bovine, canine, porcine, chimpanzee, cynomolgus monkey, baboon, Old World monkey, caprine, equine, ungulate, or feline RPE cell.

In one embodiment, the RPE cell may be differentiated from one or more pluripotent cells. In another embodiment, the pluripotent cells may be selected from the group consisting of induced pluripotent stem (iPS) cells, embryonic stem (ES) cells, blastomeres, morula cells, embroid bodies, adult stem cells, hematopoietic stem cells, fetal stem cells, mesenchymal stem cells, postpartum stem cells, multipotent stem cells, and embryonic germ cells. In a further embodiment, the pluripotent stem cell may be an embryonic stem cell. In a still further embodiment, the pluripotent stem cell may be a human embryonic stem cell.

In one embodiment, the collected cells may comprise differentiated cells. In another embodiment, the collected cells may comprise RPE cells. In another embodiment, the collected cells may consist of RPE cells. In a further embodiment, the collected cells may comprise differentiated cells and essentially no undifferentiated cells. In another embodiment, the collected cells may comprise RPE cells and essentially no other differentiated cells. In yet another embodiment, the collected cells may comprise RPE cells and essentially no undifferentiated cells. In another embodiment, the collected cells may comprise RPE cells and no pluripotent stem cells. In still another embodiment, the collected cells may comprise RPE cells and essentially no embryonic stem cells. In one embodiment, the collected cells comprise viable cells. In another embodiment, the collected cells consist of viable cells. In a further embodiment, the collected cells do not comprise any undifferentiated cells.

In one embodiment, the planar carrier may be a culture dish.

In one embodiment, the minimum specified distance between a viable cell and a detected undesired cell may be may be selected from the group consisting of 1, 2, 3, 4, 5, 6, 7, 8, 9, 10, 15, 16, 17, 18, 19, 20, 30, 40, 50, 60, 70, 80, 90, 100, 150, or 200 micrometers; 1, 2, 3, 4, 5, 6, 7, 8, 9, 10, 15, or 20 cell widths; and 1, 2, 3, 4, 5, 6, 7, 8, 9, 10, 15, or 20 layers of surrounding cells. In another embodiment, the specified distance may be at least about 1, 2, 3, 4, 5, 6, 7, 8, 9, micrometers (microns). In a further embodiment, the specified distance may be at least about 1-2 micrometers.

In one embodiment, the minimum specified distance between a differentiated cell and a detected undesired cell may be may be selected from the group consisting of 1, 2, 3, 4, 5, 6, 7, 8, 9, 10, 15, 16, 17, 18, 19, 20, 30, 40, 50, 60, 70, 80, 90, 100, 150, or 200 micrometers; 1, 2, 3, 4, 5, 6, 7, 8, 9, 10, 15, or 20 cell widths; and 1, 2, 3, 4, 5, 6, 7, 8, 9, 10, 15, or 20 layers of surrounding cells. In another embodiment, the specified distance may be at least about 1, 2, 3, 4, 5, 6, 7, 8, 9, 10 micrometers (microns). In a further embodiment, the specified distance may be at least about 1-2 micrometers.

In one embodiment, the minimum specified distance between a RPE cell and a detected undifferentiated pluripotent stem cell may be may be selected from the group consisting of 1, 2, 3, 4, 5, 6, 7, 8, 9, 10, 15, 16, 17, 18, 19, 20, 30, 40, 50, 60, 70, 80, 90, 100, 150, or 200 micrometers; 1, 2, 3, 4, 5, 6, 7, 8, 9, 10, 15, or 20 cell widths; and 1, 2, 3, 4, 5, 6, 7, 8, 9, 10, 15, or 20 layers of surrounding cells. In another embodiment, the specified distance may be at least about 1, 2, 3, 4, 5, 6, 7, 8, 9, 10 micrometers (microns). In a further embodiment, the specified distance may be at least about 1-2 micrometers.

In one embodiment, the minimum specified distance between an RPE cell and a detected undesired cell may be selected from the group consisting of 1, 2, 3, 4, 5, 6, 7, 8, 9, 10, 15, 16, 17, 18, 19, 20, 30, 40, 50, 60, 70, 80, 90, 100, 150, or 200 micrometers; 1, 2, 3, 4, 5, 6, 7, 8, 9, 10, 15, or 20 cell widths; and 1, 2, 3, 4, 5, 6, 7, 8, 9, 10, 15, or 20 layers of surrounding cells. In another embodiment, the specified distance may be at least about 1, 2, 3, 4, 5, 6, 7, 8, 9, 10 micrometers (microns). In a further embodiment, the specified distance may be at least about 1-2 micrometers.

In another embodiment, the laser light may be produced from a laser selected from the group consisting of argon ion lasers, diode lasers, dye lasers, excimer lasers, fiber lasers, free electron lasers, krypton ion lasers, Nd: YAG lasers, Nd: YVO₄ lasers, and solid-state bulk lasers. In another embodiment, the laser light may be ultraviolet light. In another embodiment, the laser light may be provided as pulses having a duration between about 100 μs and about 3000 μs. In a further embodiment, the laser light may be produced from the STILETTO® laser system.

In one embodiment, the methods described herein may be conducted under sterile conditions. In another embodiment, the method may further comprise further comprising culturing the isolated viable cell.

In an embodiment, the method may further comprise at least one additional round of laser isolation, each additional round of laser isolation comprising isolating said cell from a cell population resulting from the preceding round of laser isolation by the method according to any one of the preceding claims.

In another embodiment, the method may further comprise at least one additional rounds of laser isolation, each additional round of laser isolation comprising isolating desired cells from a cell population resulting from the preceding round of laser isolation by the method according to any one of the preceding claims.

In a yet a still further embodiment, the invention provides a purified population of RPE cells produced by a method described herein.

In a still further embodiment, the invention provides a method of preventing or treating a disease of the retina comprising providing RPE cells produced by the method of the forgoing claims; and introducing said RPE cells into the eye of an affected individual. In one embodiment, the disease of the retina may be selected from the group consisting of retinal detachment, retinal dysplasia, retinal atrophy, choroideremia, diabetic retinopathy, macular degeneration, age-related macular degeneration, retinitis pigmentosa, and Stargardt's Disease (fundus flavimaculatus). In another embodiment, the cells may be provided in a suspension, matrix, or scaffold.

In one embodiment, the pluripotent stem cells are embryonic stem cells, induced pluripotent stem (iPS) cells, single blastomeres, morula cells, embroid bodies, adult stem cells, hematopoietic cells, fetal stem cells, mesenchymal stem cells, postpartum stem cells, multipotent stem cells, or embryonic germ cells. In another embodiment, the pluripotent stem cells may be mammalian pluripotent stem cells. In still another embodiment, the pluripotent stem cells may be human pluripotent stem cells including but not limited to human embryonic stem (hES) cells, human induced pluripotent stem (iPS) cells, human adult stem cells, human hematopoietic stem cells, human fetal stem cells, human mesenchymal stem cells, human postpartum stem cells, human multipotent stem cells, or human embryonic germ cells. In another embodiment, the pluripotent stem cells may be a hES cell line listed in the European Human Embryonic Stem Cell Registry—hESCreg.

### BRIEF DESCRIPTION OF THE DRAWINGS

FIG. 1 depicts an RPE cluster with a clear boundary between RPE and non-RPE cells where the RPE cells may be identified by morphology and/or pigmentation.

FIG. 2 depicts an exemplary laser selection area completely inside an RPE cluster that may be dissected using the methods described herein.

FIG. 3 depicts an RPE cluster with a clear boundary between RPE and non-RPE cells where the RPE cells may be identified by morphology and/or pigmentation.

FIG. 4 depicts an RPE cluster isolated using collagenase followed by manual selection of pigmented clusters after 4 days in culture (40× magnification).

FIG. 5 depicts an RPE cluster isolated using laser microdissection after 4 days in culture (40× magnification).

FIG. 6 depicts an RPE cluster isolated via manual colony picking after 4 days in culture (40× magnification).

### DETAILED DESCRIPTION OF THE INVENTION

The invention relates to methods for isolation of viable cells using laser microdissection. In particular, the laser microdissection methods described herein may be used to isolate desired cells from a diverse starting population (e.g., a mixed population of cells differentiated from embryonic stem (ES) cells.) The invention provides methods comprising laser microdissection that may produce a substantially pure population of isolated cells (e.g., populations with few or no undesired cell types present). The laser microdissection methods may produce a substantially pure population of isolated cells which may be more pure than populations produced by manual colony picking or chemical separation methods (e.g., collagenase treatment). The substantially pure populations may be suitable for cell transplantation or other therapeutic uses because they contain few or no undesired cells. Surprisingly, it has been found laser microdissection may be used to isolate a pure population of desired cells from a heterogeneous population (e.g., differentiated cells purified from a heterogeneous population including non-differentiated and differentiated cells).

### DEFINITIONS

Unless defined otherwise, all technical and scientific terms used herein have the same meaning as those commonly understood by one of ordinary skill in the art to which this invention belongs. Although methods and materials similar or equivalent to those described herein can be used in the invention or testing of the present invention, suitable methods and materials are described below. The materials, methods and examples are illustrative only, and are not intended to be limiting.

As used in the description herein and throughout the claims that follow, the meaning of “a,” “an,” and “the” includes plural reference unless the context clearly dictates otherwise.

“Embryo” or “embryonic,” as used herein refers broadly to a developing cell mass that has not implanted into the uterine membrane of a maternal host. An “embryonic cell” may be a cell isolated from or contained in an embryo. This also includes blastomeres, obtained as early as the two-cell stage, and aggregated blastomeres.

“Embryonic stem cells” (ES cells), as used herein, refers broadly to cells derived from the inner cell mass of blastocysts or morulae that have been serially passaged as cell lines. The ES cells may be derived from fertilization of an egg cell with sperm or DNA, nuclear transfer, parthenogenesis, or by means to generate ES cells with homozygosity in the HLA region. ES cells may also refer to cells derived from a zygote, blastomeres, or blastocyst-staged mammalian embryo produced by the fusion of a sperm and egg cell, nuclear transfer, parthenogenesis, or the reprogramming of chromatin and subsequent incorporation of the reprogrammed chromatin into a plasma membrane to produce a cell. Embryonic stem cells, regardless of their source or the particular method used to produce them, may be identified based on the: (i) ability to differentiate into cells of all three germ layers, (ii) expression of at least Oct-4 and alkaline phosphatase, and (iii) ability to produce teratomas when transplanted into immunocompromised animals.

“Embryo-derived cells” (EDC), as used herein, refers broadly to morula-derived cells, blastocyst-derived cells including those of the inner cell mass, embryonic shield, or epiblast, or other pluripotent stem cells of the early embryo, including primitive endoderm, ectoderm, and mesoderm and their derivatives. “EDC” also including blastomeres and cell masses from aggregated single blastomeres or embryos from varying stages of development, but excludes human embryonic stem cells that have been passaged as cell lines.

“Isolated,” as used herein, describes cells that are substantially free of at least one protein, molecule, or other impurity that is found in its natural environment (e.g., “substantially purified”.) The term “isolated” may be used interchangeably with “purified.”

“Laser microdissection system,” as used herein, refers broadly to any method using a laser to isolate cells from a sample, including but not limited to laser capture microdissection (LCM), laser microdissection and pressure catapulting (LMPC), laser microdissection (LMD), and laser-assisted microdissection (LMD or LAM).

“Mature RPE cell” and “mature differentiated RPE cell,” as used herein, may be used interchangeably throughout to refer broadly to changes that occur following initial differentiating of RPE cells. Specifically, although RPE cells may be recognized, in part, based on initial appearance of pigment, after differentiation mature RPE cells may be recognized based on enhanced pigmentation.

“Multipotent cell,” as used herein refers broadly to any cell that has the potential to give rise to cells from multiple lineages within a cell type (e.g., a hematopoietic cell—a blood cell that can develop into several types of blood cells).

“Pigmented,” as used herein refers broadly to any level of pigmentation, for example, the pigmentation that initial occurs when RPE cells differentiate from ES cells. Pigmentation may vary with cell density and the maturity of the differentiated RPE cells. The pigmentation of a RPE cell may be the same as an average RPE cell after terminal differentiation of the RPE cell. The pigmentation of a RPE cell may be more pigmented than the average RPE cell after terminal differentiation of the RPE cell. The pigmentation of a RPE cell may be less pigmented than the average RPE cell after terminal differentiation.

“Pluripotent stem cell,” as used herein, refers broadly to a cell capable of prolonged or virtually indefinite proliferation in vitro while retaining their undifferentiated state, exhibiting normal karyotype (e.g., chromosomes), and having the capacity to differentiate into all three germ layers (i.e., ectoderm, mesoderm and endoderm) under the appropriate conditions.

“Pluripotent embryonic stem cells,” as used herein, refers broadly cells that: (a) are capable of inducing teratomas when transplanted in immunodeficient (SCID) mice; (b) are capable of differentiating to cell types of all three germ layers (e.g., ectodermal, mesodermal, and endodermal cell types); and (c) express at least one molecular embryonic stem cell markers (e.g., express Oct 4, alkaline phosphatase, SSEA-3 surface antigen, SSEA-4 surface antigen, NANOG, TRA-1-60, TRA-1-81, SOX2, REX1).

“RPE cell,” “differentiated RPE cell,” “ES-derived RPE cell,” and as used herein, may be used interchangeably throughout to refer broadly to an RPE cell differentiated from a pluripotent stem cell using a method of the invention. The term is used generically to refer to differentiated RPE cells, regardless of the level of maturity of the cells, and thus may encompass RPE cells of various levels of maturity. RPE cells may be visually recognized by their cobblestone morphology and the initial appearance of pigment. RPE cells may also be identified molecularly based on substantial lack of expression of embryonic stem cell markers such as Oct-4 and NANOG, as well as based on the expression of RPE markers such as RPE-65, PEDF, CRALBP, and bestrophin. Thus, unless otherwise specified, RPE cells, as used herein, refers to RPE cells differentiated in vitro from pluripotent stem cells.

### Laser Microdissection

Laser capture microdissection (LCM) (a.k.a., microdissection, laser microdissection (LMD), laser microdissection and pressure catapulting (LMPC), or laser-assisted microdissection (LMD or LAM) is a method for isolating specific cells of interest from a tissue, cell population, or organism.

Generally, a laser may be coupled to a microscope and focused onto the heterogeneous cell population (e.g., tissue) in a culture dish. By movement of the laser by optics or the stage the focus follows a trajectory which may be predefined by the user. This trajectory, the element, may then be cut out and separated from the adjacent cells (e.g., tissue.) After the cutting process, a collection process may be used to remove the target cells from the sample.

The laser microdissection systems may employ a variety of lasers including but not limited to UV lasers (e.g., UV-A laser (˜355 nm)). Further, various computer systems for laser dissection are known in the art and may be used in the methods described herein. For example, the Stiletto® laser dissection system from Hamilton Thorne, Olympus SmartCut® laser microdissection system, CellCut® laser microdissection system with MMI CapLift®, or AutoPix® laser capture microdissection system, ArcturusXT® laser capture microdissection system may be used. Additionally, any one or all of the steps of the methods described herein may be automated. Further, any one or all of the steps of the methods described herein may be conducted under sterile conditions.

In one aspect, the invention provides a method for isolating differentiated cells from a heterogeneous population of cells comprising placing a culture dish containing said heterogeneous cell population on a microscope coupled to a laser dissection system, selecting differentiated cells for isolation, excising the differentiated cells, and collecting said differentiated cells.

In one aspect, the disclosure provides a method of isolating viable cells comprising providing a planar carrier, placing a heterogeneous cell population comprising differentiated cells; selecting at least one cells to be isolated; excising the cells, thereby severing the connections between the selected cells and adjacent cells or other materials; and separating the selected cells from the planar carrier, thereby isolating the selected cells. The desired cells are preferably ocular cells, and most preferably RPE. The laser ablating may be automated, for example, the user selects the cells to be isolated and provides information to a computer running a laser cutting program (e.g., mmi SmartCut Plus, mmi CellCut®). A high precision, motorized XY-stage may be controlled through computer mouse or keyboard. The program may comprise an overview that allows for navigation within the culture dish to facilitate selection of the desired cells. Several positions of the stage may be stored for returning to an area of interest. The program may comprise a drawing tool where for marking the cutting path, the user may choose between free hand drawing and geometric figures such as circles, squares and ellipses for selecting desired cells. This allows the user to mark objects over the entire slide area and these objects will the selected area may be cut automatically by the computer. The size of the circles, squares and ellipses may be chosen by the user and be copied via use of a computer. In one embodiment, automation of the methods described herein allows for the isolation of large amounts of highly pure populations RPE cells differentiated from ES cells under sterile conditions in a reduced period of time (compared to manual or chemical selection of RPE cells). For example, the laser isolation method described herein may be fully automated to allow for the isolation of RPE cells without manual or chemical selection of RPE cells. This allows for significant savings in cost (including labor) and time (e.g., isolated the cells in a matter of hours instead of days or weeks).

In another aspect, laser microdissection and pressure catapulting (LMPC) may be used. In LMPC, a heterogeneous population of cells in a culture dish may be placed directly on top of a thermoplastic polyethyelene napthalate membrane that covers the culture dish. The membrane acts as a support (scaffolding) to allow for catapulting relatively large amounts of intact material at a time. A focused laser beam may be used to cut out an area of the membrane and corresponding biological sample, and the beam may be then defocused and the energy used to catapult the membrane and material from the slide. A motorized robotic (e.g., RoboMover) stage may be used to move the sample through the laser beam path to allow the user to control the size and shape of the area to be cut. The catapulted sample may be captured in an aqueous media positioned directly above the cut area. See Kuhn, et al. (2007) Am J Phyiol. Heart Circ. Physiol. 292: H1245-H1253, H1245.

The starting population of cells may be differentiated from any pluripotent cells. For example, the pluripotent cells may be embryonic stem cells, induced pluripotent stem (iPS) cells, single blastomeres, morula cells, embroid bodies, adult stem cells, hematopoietic cells, fetal stem cells, mesenchymal stem cells, postpartum stem cells, multipotent stem cells, or embryonic germ cells. In another embodiment, the pluripotent stem cells may be mammalian pluripotent stem cells. In still another embodiment, the pluripotent stem cells may be human pluripotent stem cells including but not limited to human embryonic stem (hES) cells, human induced pluripotent stem (iPS) cells, human adult stem cells, human hematopoietic stem cells, human fetal stem cells, human mesenchymal stem cells, human postpartum stem cells, human multipotent stem cells, or human embryonic germ cells. In another embodiment, the pluripotent stem cells may be a hES cell line listed in the European Human Embryonic Stem Cell Registry—hESCreg. Also, the pluripotent stem cells may be human embryonic stem cells (hES cells), human induced pluripotent stem (iPS) cells, or embryonic stem cells of another species. Further, the pluripotent stem cells of (a) may be genetically engineered. The starting population of cells may comprise an embryoid body. For example, a pluripotent stem cell may be differentiated to produce a heterogeneous population comprising at least one differentiated cell. The differentiated cell may then be isolated using laser microdissection methods described herein. Further, the isolated cell may be further cultured to expand the isolated population or to confirm the purity of the isolated cells (e.g., culture the isolated cell to confirm the absence of undesired cells).

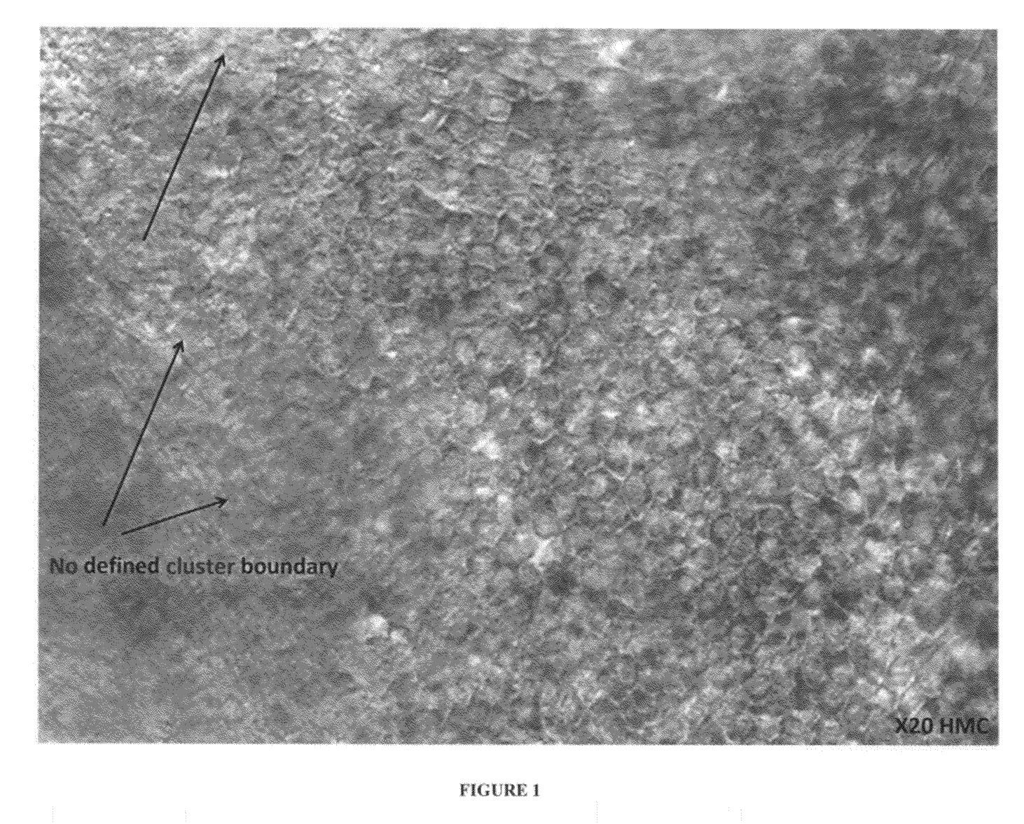

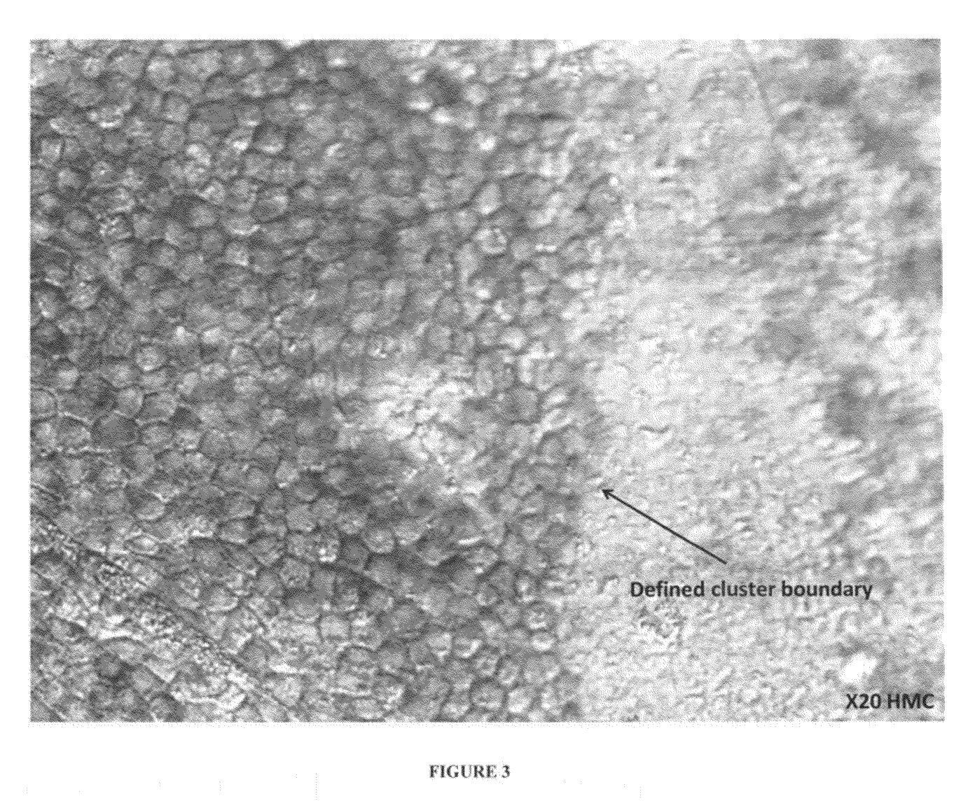

The population of differentiated cells may be produced by culturing ES cells using the methods disclosed in U.S. Pat. Nos. 7,795,025; 7,794,704; 7,736,896; U.S. patent application Ser. No. 12/682,712, International Patent Application No. PCT/US2010/57056, and WO 2009/051671. For example, ES cells may be cultured as a multilayer population or embryoid body for a sufficient duration for the appearance of pigmented epithelial cells or other differentiated cell types, which may then be isolated and further cultured. After differentiation, the ES cell population produces a heterogeneous population of cells comprising both undifferentiated ES cells and differentiated cells (e.g., RPE cells). The differentiated cells may be distinguished from the undifferentiated ES cells and other differentiated cells (e.g., non-RPE cells) in the heterogeneous cell population based on color, characteristic shape, size, cellular markers, or cellular functions (e.g., enzymatic markers). For example, in a heterogeneous population of cells comprising ES cells and RPE cells, the RPE cells are selected for isolation based on morphological characteristics including but not limited to pigmentation, a characteristic cobblestone, epithelial appearance (mottled appearance), or RPE cells markers. The methods described herein may comprise differentiating RPE cells from a cell population of ES cells. The differentiated RPE cells may form tightly-packed pigmented colonies. These colonies may be selected for isolation using the laser microdissection methods described herein. In one embodiment, the selection area may be totally within the pigmented differentiated RPE cell colony. In this embodiment, no undifferentiated ES cells are excised, yielding a pure population of differentiated RPE cells (e.g., no ES cells). The selection area may be defined by the boundary between the pigmented RPE cells and the undifferentiated ES cells. See, e.g., FIGS. 1 and 3. A nearly pure population of differentiated RPE cells may be isolated (e.g., essentially no ES cells or other differentiated cells). These methods may also produce RPE cells that are suitable for therapeutic use, such as treatment of macular degeneration by cell transplantation into an affected eye.

Selection of cells for laser microdissection may be generally based on detectable characteristics (e.g., presence of a cell marker, absence of a cell marker, uptake of a dye, morphology, pigmentation). The cells selected for isolation may exhibit at least one detectable characteristics indicative of a desired cell, and/or may not exhibit at least one detectable characteristics whose presence would indicate an undesired cell. For example, when differentiated cells are to be isolated from a population that arose by differentiation of ES cells, cells may be selected for isolation only if they do not include any cells exhibiting detectable characteristic(s) of ES cells.

At the time of their excision, the selected cells may be at least a minimum specified distance from any undesired cells. For example, the only cells in contact with the selected cells may be cells that exhibit detectable characteristics indicative of desired cells and/or that do not exhibit a detectable characteristic indicative of an undesired cell. As another example, the selected cells may be fully contained within an island of cells that exhibit the detectable characteristics being used to identify desired cells, and/or adjacent to cell-free spaces. The minimum specified distance may be specified in distance units (e.g., at least 10, 20, 30, 40, 50, 60, 70, 80, 90, 100, 150, or 200 micrometers), as a number of cell widths (e.g., 1, 2, 3, 4, 5, 6, 7, 8, 9, 10, 15, or 20 cell widths), and/or as a minimum layers of surrounding cells (e.g., 1, 2, 3, 4, 5, 6, 7, 8, 9, 10, 15, or 20 cells) between the selected cells and any cells that exhibit characteristics of undesired cells or that do not exhibit the characteristics being used to identify desired cells. Moreover, the distance may be at least about 1-2 μm. Such methods may provide further assurance that the isolated cells are free from undesired cells, e.g., due to the possibility that an individual undesired cell may be more difficult to detect within a group of desired cells, and such an undesired cell may be more likely to be located near the periphery of an island of desired cells than internally.

Optionally, laser microdissection may be utilized for multiple iterations, wherein cells are isolated by laser microdissection and optionally cultured, and the resulting cell population may be subjected to laser microdissection. Use of multiple iterations may provide even greater assurance that undesired cells are not present, and may also help ensure phenotypic stability and uniformity in the resulting population of cultured cells. The cell(s) isolated during each round may also be selected on the basis of a trait (e.g., level of expression of a particular gene) to facilitate isolation of a more desirable cell population. Cells may be isolated based on the same trait or different traits during successive iterations.

The cells to be isolated are typically provided on a planar carrier. The planar carrier used may be of any type, so long as it allows light to pass through. Typically the support may be optically clear. In a preferred embodiment the support may be polystyrene which may be optionally tissue-culture treated polystyrene. Other suitable supports may include glass (e.g., a glass slide or cover slip), polyethylene terephthalate, polycarbonate, polyethylene, polypropylene Particularly preferred carriers include microtiter plates (e.g., 6-, 12-24-, 96-, 384-, and 1536-well plates)

The cutting out of a target area may be preferably performed under microscopic view. Alternatively, or in addition, the target area may be visualized through use of an image recording device, such as a CCD camera, which may be used to generate an image of the material located on the carrier, and display it on a display device. This image may be superimposed with a user interface of the laser microdissection system to facilitate selecting the objects to be processed with the laser beam.

Preferably, the planar carrier may be movable within the microscopic view, thereby facilitating isolation of desired cells from various portions of the planar carrier. For example, the planar carrier may be affixed to a moveable stage (e.g., an X-Y or X-Y-Z stage). Movement of the planar carrier may be performed manually or may be automated, for example, driven by a computer-controlled stepper motor. For example, automated movement of the planar carrier during laser ablation may be used to move the target areas into the laser beam path. Exemplary moveable stages are available from Prior Scientific (e.g., the PROSCAN® product lines).

The laser light may be typically focused to a small diameter and applied to the sample, preferably from the bottom side of the support, along a target position on the biological preparation, thereby cutting out the biological preparation.

The laser light may be of any wavelength that may be used to excise cells or other materials in target areas adjacent to the selected cells while preferably retaining the viability of adjacent non-irradiated cells. In a preferred embodiment, the laser light may be ultraviolet light, e.g., having a wavelength less than about 400 nm. Preferably, the wavelength may be between 200 and 400 nm, such as near-UV (between 400 and 300 nm), middle-UV (between 300 and 200 nm), UVA (between 400 and 320 nm), UVB (between 320 and 280 nm) or UVC (between 280 and 200 nm). Known ultraviolet lasers and methods of producing ultraviolet laser light may be utilized, including argon ion lasers; diode lasers (e.g., based on gallium nitride); dye lasers; excimer lasers (including F₂, ArF, KrF, XeBr, or XeCl, XeF); fiber lasers such as neodymium-doped fluoride fiber lasers; free electron lasers; krypton ion lasers; lasers producing wavelengths longer than ultraviolet and incorporating non-linear frequency conversion (such as an Nd: YAG or Nd: YVO₄ laser coupled to two successive frequency doublers); and solid-state bulk lasers including cerium-doped crystals such as Ce³⁺: LiCAF or Ce³⁺: LiLuF₄ (which may optionally be pumped with nanosecond pulses from a frequency-quadrupled Q-switched laser). Further exemplary laser systems are described in U.S. Pat. Nos. 4,641,912; 4,773,414; 4,784,135; 4,785,806; 5,144,630; 5,146,465; 5,237,576; 5,742,626; 5,745,284; and 7,277,220.

The laser light may be delivered in pulses or continuously. For example, the laser pulse length may be between 100 μs and 3000 μs, or shorter or longer pulse durations may also be utilized. The duration and frequency of laser pulses may be adjusted appropriately in such a manner that a required amount of energy may be directed to a target area to be cut. Preferably, the laser pulse duration and frequency are sufficient to sever connections between the selected cells and surrounding material, while retaining viability of the cells to be isolated.

In a preferred embodiment, the laser module may be combined into a single unit with an objective (e.g., a 20× objective), for example, as a single compact turret mounted unit. A particularly preferred laser module may be the STILETTO® laser system available from Hamilton Thorne Ltd. (Beverly, Mass.).

The method may be performed manually or may include use of an automated system. An automated system may perform at least one or all of the steps of the method without the need for human intervention or with human supervision or intervention. For example, based on the presence of detectable characteristics an automated system may suggest cells for isolation and/or suggest target areas for laser ablation, and a human operator may accept, modify, or reject the suggestions by the automated system.

Several ways for collecting cells which have been isolated from a heterogenous population on a microscope slide (e.g., culture dish) are known in the art. For example, the isolated cells may be collected by pipette, washing, or laser pressure catapult.

For example, the excised cells may be catapulted by a photonic cloud into a microcentrifuge tube cap. The cells may be attached to a cap lined with a thermoplastic film that forms a protrusion when hit with a laser pulse. The protrusion closes the gap between the cells and the film. Lifting the cap may remove the target cells and keep them attached to the cap. The cap may be then placed in a microcentrifuge tube for processing. This cap method may be used in conjunction with cutting cells from a tissue section and then attaching them to a cap. The cells may be propelled using an electrostatic force toward a film, and then the film may be pushed inside a microcentrifuge tube for collection.

In a cell ablation method, live cells in a sterile culture dish may be covered with a light absorbing film. The laser may cut around the cells of interest under the film and, when the film may be removed, the cells stay in the culture dish and the unwanted cells (e.g., undifferentiated cells) come off with the film. This method is referred to as “cell ablation” because it removes the unwanted cells from the culture and the remaining cells may be washed and re-cultured. See Bancroft & Gamble (2008) Theory and Practice of Histological Techniques, page 575.

In a laser catapult method, the sample may be catapulted from a culture dish by a defocused U.V laser pulse that generates a photonic force propelling the material off the dish. This is also referred to as Laser Micro-dissection Pressure Catapulting (LMPC) and the cells may be sent upward (e.g., up to several mm) to a collection vessel (e.g., microfuge tube cap) containing buffer or a specialized material in the tube cap that the cells may adhere to. This active catapulting process avoids some of the static problems when using membrane-coated slides. See, e.g., Zeiss PALM MicroBeam; U.S. Pat. Nos. 5,689,109; 5,998,129; and 6,930,714. Another similar LCM process cuts the sample from above and the sample drops via gravity into a capture device below the sample. See Leica Microsystems Laser Microdissection System. Further, the excised cells may be collected by pipetting, or manual picking of the excised cells after they are excised from the heterogeneous population in the laser microdissection system.

Further, the methods described herein may be conducted under sterile conditions. For example, the methods described herein may be conducted in accordance with Good Manufacturing Practices (GMP) (e.g., the cultures are GMP-compliant) and/or current Good Tissue Practices (GTP) (e.g., the cultures may be GTP-compliant.)

### Isolated Cell Populations

The present invention provides purified preparations of desired cells, preferably differentiated cells isolated from a heterogeneous population comprising differentiated and non-differentiated cells (e.g., RPE cells isolated from a heterogeneous population of RPE cells, ES cells, and differentiated cells). The desired cells isolated by the methods described herein may be substantially free of at least one protein, molecule, or other impurity that is found in its natural environment (e.g., “isolated”.) For example, the methods described herein may provide isolated RPE cells, substantially purified populations of RPE cells, and pharmaceutical preparations comprising RPE cells.

The desired cells isolated by the laser microdissection methods described herein may be differentiated from a pluripotent stem cell or a multipotent cell. For example, a desired cell may be differentiated from a pluripotent stem cells including but not limited to embryonic stem cells, induced pluripotent stem (iPS) cells, adult stem cells, hematopoietic cells, fetal stem cells, mesenchymal stem cells, postpartum stem cells, multipotent stem cells, or embryonic germ cells. The desired cell may be differentiated from any mammalian pluripotent cell that is capable of giving rise thereto via differentiation. The desired cell may be differentiated from a human pluripotent cells including but not limited to human embryonic stem (hES) cells, human induced pluripotent stem (iPS) cells, blastomeres or morula, embroid bodies, human adult stem cells, human hematopoietic stem cells, human fetal stem cells, human mesenchymal stem cells, human postpartum stem cells, human multipotent stem cells, or human embryonic germ cells. Further, the pluripotent stem cells may be a hES cell line listed in the European Human Embryonic Stem Cell Registry—hESCreg.

The desired cells isolated by the laser microdissection methods described herein from a heterogeneous cell population that may comprises a desired differentiated cell, differentiated cells that may not be desired, and undifferentiated cells.

The preparations may be substantially purified, with respect to non-differentiated cells, comprising at least about 75%, 80%, 85%, 90%, 91%, 92%, 93%, 94%, 95%, 96%, 97%, 98%, 99%, or 100% differentiated cells. The differentiated cell preparation may be essentially free of non-differentiated cells or consist of differentiated cells. For example, the substantially purified preparation of differentiated cells may comprise less than about 25%, 20%, 15%, 10%, 9%, 8%, 7%, 6%, 5%, 4%, 3%, 2%, or 1% non-differentiated cell type. For example, the differentiated cell preparation may comprise less than about 25%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% non-differentiated cells.

Further, RPE cell preparations isolated using the methods described herein may be substantially pure, both with respect to non-RPE cells and with respect to RPE cells of other levels of maturity. The preparations may be substantially purified, with respect to non-RPE cells, and enriched for mature RPE cells. For example, in RPE cell preparations enriched for mature RPE cells, at least about 30%, 40%, 45%, 50%, 55%, 60%, 65%, 70%, 75%, 80%, 85%, 90%, 91%, 92%, 93%, 94%, 95%, 96%, 97%, 98%, 99%, 99%, or 100% of the RPE cells are mature RPE cells. The preparations may be substantially purified, with respect to non-RPE cells, and enriched for differentiated RPE cells rather than mature RPE cells. For example, at least about 30%, 40%, 45%, 50%, 55%, 60%, 65%, 70%, 75%, 80%, 85%, 90%, 91%, 92%, 93%, 94%, 95%, 96%, 97%, 98%, 99%, or 100% of the RPE cells may be differentiated RPE cells rather than mature RPE cells.

The differentiated cell preparations isolated using the methods described herein may comprise at least about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ differentiated cells. The differentiated cell preparations isolated using the methods described herein may comprise at least about 5,000-10,000, 50,000-100,000, 100,000-200,000, 200,000-500,000, 300,000-500,000, or 400,000-500,000 differentiated cells. The differentiated cell preparation may comprise at least about 20,000-50,000 differentiated cells. Also, the differentiated cell preparation may comprise at least about 5,000, 10,000, 20,000, 30,000, 40,000, 50,000, 60,000, 70,000, 75,000, 80,000, 100,000, or 500,000 differentiated cells.

The differentiated cell preparations may comprise at least about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ differentiated cells/mL. The differentiated cell preparations may comprise at least about 5,000-10,000, 50,000-100,000, 100,000-200,000, 200,000-500,000, 300,000-500,000, or 400,000-500,000 differentiated cells/mL. The differentiated cell preparation may comprise at least about 20,000-50,000 differentiated cells/mL. Also, the differentiated cell preparation may comprise at least about 5,000, 10,000, 20,000, 30,000, 40,000, 50,000, 60,000, 75,000, 80,000, 100,000, or 500,000 differentiated cells/mL. Additionally, the differentiated cell preparation may comprise at least about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, or 1×10⁷.

The differentiated cell culture may be a substantially purified culture comprising at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% differentiated cells. The substantially purified culture may comprise at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% mature differentiated cells.

The differentiated cell cultures may be prepared in accordance with Good Manufacturing Practices (GMP) (e.g., the cultures are GMP-compliant) and/or current Good Tissue Practices (GTP) (e.g., the cultures may be GTP-compliant.)

### Retinal Pigment Epithelium (RPE) Cells

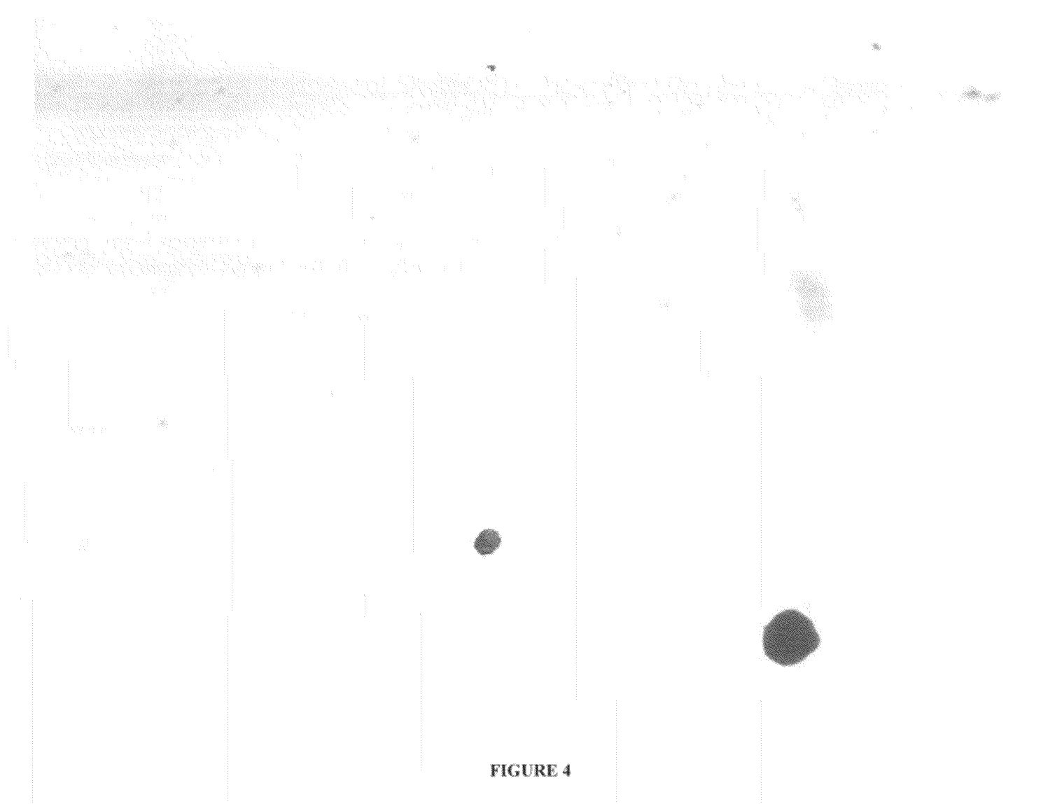

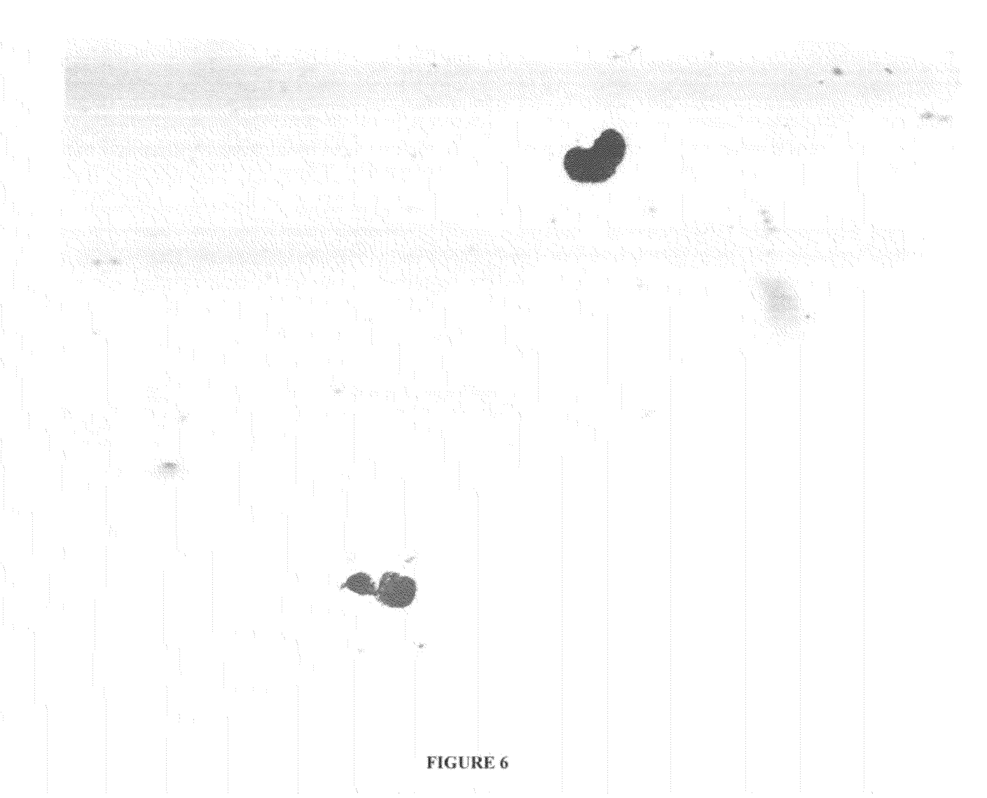

The present invention provides RPE cells that may be isolated from a heterogeneous population of cells comprising, for example, pluripotent cells, such as human embryonic stem cells or human iPSC's, and are molecularly distinct from embryonic stem cells, adult-derived RPE cells, and fetal-derived RPE cells. See, also, Liao, et al. (2010) Human Molecular Genetics 19(21): 4229-4238. The inventors surprisingly discovered that the method by which the RPE cells are isolated from a heterogeneous population of cells, for example, pluripotent stem cells from which they may be differentiated, may an important factor in determining the purity of the resulting RPE cells. The inventors found that the RPE cells produced by the methods described produced a substantially pure RPE cell population (e.g., essentially no non-RPE cells) than previous methods of isolated RPE cells. Further, the methods described herein are less labor-intensive and faster than methods using chemical agents (e.g., collagenase) or labor-intensive methods (e.g., manual colony picking). See, e.g., FIGS. 4 and 6, respectively. For example, the isolation methods described herein allow for the rapid and repeatable final RPE cell product of substantial purity (e.g., essentially no non-RPE cells). Further, the methods of isolating RPE cells described herein that avoid the inclusion of ES cells in the final RPE cell population. Thus, as ES cells are not present in any amount in populations isolated by the methods described herein, and they do not pose an unacceptable risk of contamination in the RPE cell cultures and preparations.

The cell types that may be isolated from a heterogeneous cell population by this invention include, but are not limited to, RPE cells, RPE progenitor cells, iris pigmented epithelial (IPE) cells, and other vision associated neural cells, such as internuncial neurons (e.g., “relay” neurons of the inner nuclear layer (INL)) and amacrine cells. The invention also provides methods of isolating retinal cells, rods, cones, and corneal cells as well as cells providing the vasculature of the eye from heterogeneous population. Further, the methods described herein may be used to isolated RPE cells from a heterogeneous population comprising RPE cells, pluripotent stem cells, and other non-RPE differentiated cells.

The RPE cells isolated by the methods described herein may be used for treating retinal degeneration diseases due to retinal detachment, retinal dysplasia, or retinal atrophy or associated with a number of vision-altering ailments that result in photoreceptor damage and blindness, such as, choroideremia, diabetic retinopathy, macular degeneration (e.g., age-related macular degeneration), retinitis pigmentosa, and Stargardt's Disease (fundus flavimaculatus).

The RPE cells may express at least one RPE cell marker that may be used to identify the RPE cells in a heterogenous population for isolation. For example, the RPE cells may express RPE65, PAX2, PAX6, tyrosinase, bestrophin, PEDF, CRALBP, Otx2, or MitF. Additionally, the RPE cells may show elevated expression levels of alpha integrin subunits 1-6 or 9 as compared to uncultured RPE cells or other RPE cell preparations. The RPE cells described herein may also show elevated expression levels of alpha integrin subunits 1, 2, 3, 4, 5, or 9. The RPE cells described herein may be cultured under conditions that promote the expression of alpha integrin subunits 1-6. For example, the RPE cells may be cultured with integrin-activating agents including but not limited to manganese and the activating monoclonal antibody (mAb) TS2/16. See Afshari, et al. Brain (2010) 133(2): 448-464. The RPE cells may be plated on laminin (1 μg/mL) and exposed to Mn²⁺ (500 μM) for at least about 8, 12, 24, 36, or 48 hours.

Table 1 describes characteristics of the RPE cells that may be used to identify or characterize the RPE cells. In particular, the RPE cells may exhibit a normal karyotype, express RPE markers, and not express hES markers. These markers may be used to identify RPE cells in a heterogeneous population for them to be isolated using the methods described herein.

TABLE 1
Parameters of RPE cells

| Parameter | Specification for RPE Cells |
|---|---|
| Karyotype | Normal |
| | (e.g., 46 chromosomes for |
| | human RPE cells) |
| Morphology at harvest | Normal cellular morphology, |
| | medium pigmentation |
| Post-thaw Viable Cell Count | ≧70% |
| qPCR Testing-Presence of RPE Markers | Present |
| Bestrophin | |
| RPE-65 | |
| CRALBP | |
| PEDF | |
| PAX6 | |
| MITF | |
| qPCR Testing-Absence of hES Markers | Absent |
| Oct-4 | |
| NANOG | |
| Rex-1 | |
| Sox2 | |
| Immunostaining-Presence of RPE Markers | Present |
| Bestrophin | |
| CRALBP | |
| PAX6 | |
| MITF | |
| ZO-1 | |
| Immunostaining-Absence of hES markers | Absent |
| Oct-4 | |
| Alkaline Phosphatase | |

The distinct expression pattern of mRNA and proteins in the RPE cells of the invention constitutes a set of markers that separate these RPE cells from cells in the art, such as hES cells, ARPE-19 cells, and fetal RPE cells. Specifically, these cells are different in that they may be identified or characterized based on the expression or lack of expression, which may be assessed by mRNA or protein level, of at least one marker. For example, the RPE cells may be identified or characterized based on expression or lack of expression of at least one marker listed in Table 1. See also Liao, et al. (2010) Human Molecular Genetics 19(21): 4229-38. The RPE cells may also be identified and characterized, as well as distinguished from other cells, based on their structural properties. Thus, the RPE cells described herein expressed multiple genes that were not expressed in hES cells, fetal RPE cells, or ARPE-19 cells. See WO 2009/051671; See also Dunn, et al. (1996) Exp Eye Res. 62(2): 155-169.

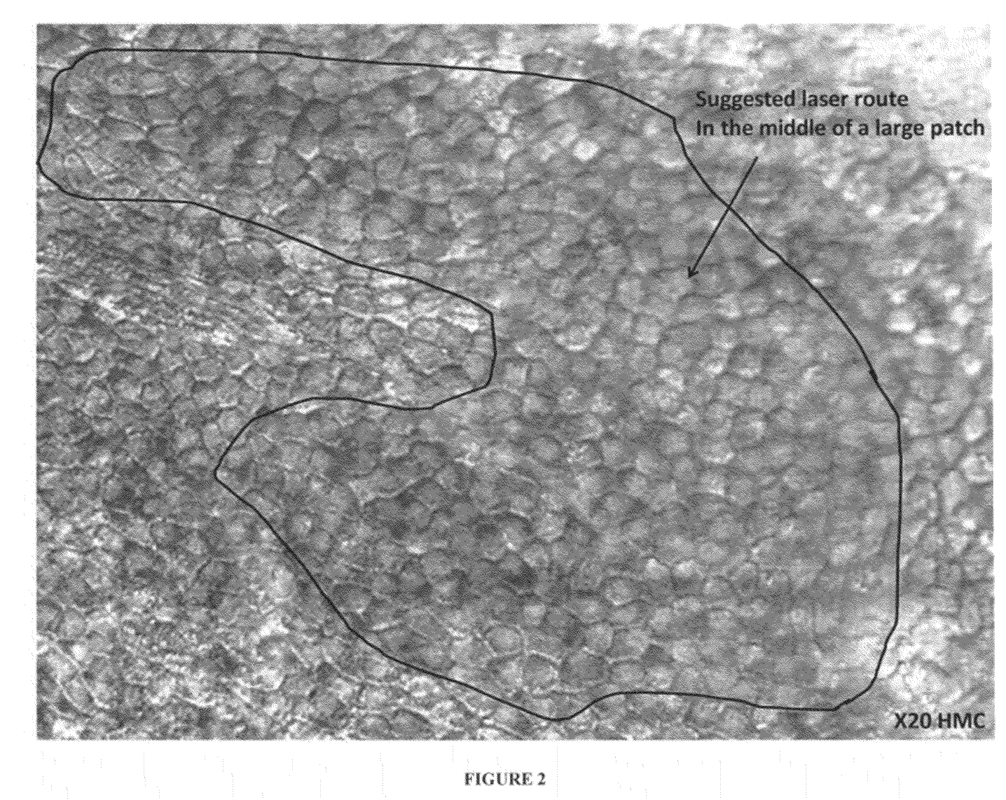

The RPE cells described herein may also be identified and characterized based on the degree of pigmentation of the cell. Pigmentation post-differentiation may be not indicative of a change in the RPE state of the cells (e.g., the cells are still differentiated RPE cells). Rather, the changes in pigment post-differentiation correspond to the density at which the RPE cells are cultured and maintained. Mature RPE cells have increased pigmentation that accumulates after initial differentiation. For example, the RPE cells described herein may be mature RPE cells with increased pigmentation in comparison to differentiated RPE cells. Differentiated RPE cells that are rapidly dividing are lightly pigmented or unpigmented. However, when cell density reaches maximal capacity, or when RPE cells are specifically matured, RPE take on their characteristic phenotypic hexagonal shape and increase pigmentation level by accumulating melanin and lipofuscin. As such, initial accumulation of pigmentation serves as an indicator of RPE differentiation and increased pigmentation associated with cell density serves as an indicator of RPE maturity. For example, the RPE cells may be pigmented. For example, the RPE cell may be derived from a human embryonic stem cell, which cell may be pigmented and expresses at least one gene that may be not expressed in a cell that may be not a human retinal pigmented epithelial cell. Further, RPE cells may be derived from differentiation of embryonic stem cells to produce a heterogeneous population of embryonic stem cells and RPE cells. The RPE cells may be morphologically distinguished from the embryonic cells on the basis of color (e.g., pigmentation), characteristic shape, size, RPE-specific cell markers, and the absence of ES-specific cell markers. For example, RPE cells may display a characteristic mottled appearance and cluster to form dark, pigmented clusters of RPE cells surrounded by undifferentiated, less pigmented ES cells (e.g., dark clusters of RPE cells surrounded by translucent ES cells when examined by light microscopy). See FIGS. 1 and 3. The inventors surprisingly discovered that laser microdissection method may select an area completely within the dark cluster of RPE cells and thus exclude all contaminating cells of any other type (e.g., ES, ES cell progeny, other differentiated cells). See FIG. 2. This unexpectedly allowed for the isolation of a large pure populations of RPE cells differentiated from ES cells under sterile conditions in a reduced period of time (compared to manual or chemical selection of RPE cells). Furthermore, this invention allowed for the isolation of ultra-pure populations of RPE cells differentiated from ES cells under sterile conditions in a reduced period of time (e.g., comprising no ES cells compared to manual or chemical selection of RPE cells).

Moreover, after the culture containing RPE clusters is treated with collagenase, the current approach, the desired cells may be difficult to differentiate because the morphology of clusters in suspension is very different from their cobblestone appearance (and different for other cell types that could be of interest), so the operator has to rely on brown color as primary assessment criteria. However, very dark pigmented cells may show poor attachment and low survival. Lightly pigmented cells may be discarded because it is often difficult to differentiate between light and no pigmentation when using a dissecting microscope (each cluster would need to be examined individually and from different sides at a high power microscope which limits its use for cluster harvesting—even if it could be built into a biosafety hood, such thorough examination would be time-prohibitive for large scale cell harvest). As a result, unpigmented clusters may be discarded as well. At the same time, when a culture is examined prior to harvesting, it has visible large fields where one could see the cobblestone morphology spreading form dark pigmented to lightly or non-pigmented areas, and with the laser help those lightly pigmented cells may also be harvested. Thus, the laser isolation methods described herein provide a method allowing an operator to identify and isolate less heavily pigmented RPE cells form a heterogeneous population in an efficient and rapid manner (as compared to conventional methods).

Additionally, laser microdissection may be used to isolate RPE clusters that may contain contaminating cells on the periphery. The clusters comprising contaminating cells may be isolated using laser dissection and then allowed to attach to a tissue culture plate. The clusters may then be further cultured, inspected, and laser dissected a second time. Further, the methods described herein may be used to isolated RPE clusters which cannot be easily excised by the laser from the original monolayer. RPE clusters which may not be cleanly excised by laser microdissection from the original monolayer may be isolated and subsequently treated with a collagenase digestion to further purify the cells (e.g., remove unwanted undifferentiated or other non-RPE cell types). Again, these RPE cell isolates may be further cultured to allow for confirmation of their purity and desired phenotype.

Another aspect of the invention involves the isolation of RPE cells and other desired cell types from a heterogeneous population of cells differentiated from ES cells. The laser microdissection methods described herein may be used when more than one type of cell may be isolated, but one cell type would be lost if the monolayer was digested (e.g., collagenase digestion). For example, in the culture of hES cells en route towards RPE differentiation, there are, for instance, neural rosettes which may potentially produce RPE as well as other cell types of the neural lineage. Using the laser microdissection methods described herein it may be possible to excise the desired cells without disturbing RPE clusters and vice versa, remove the RPE cells, and leave the other cell types (e.g., neural rosettes) to allow for further differentiation.

Further, the inventors developed a method of isolating cells of interest based on surface marker expression. Immunostaining requires either a fluorescence microscope with laser or color reaction. However, fluorescence may be harmful for the cells, even evaluation of the culture before the laser is given the coordinates may be damaging, and color products do not keep the cells alive. To avoid these problems, the inventors used manual selection of the cells after incubation with magnetic beads-conjugated antibodies (or the same sandwich indirectly, antibodies followed by the beads). In particular, DYNAL® beads may be used and are considerably large compared to beads used in the MACS system and thus the beads on the cell surface are easily identified. This method may be used instead of the fluorescent tag for visualization, and after the selection the beads may not interfere with the cells' growth and may be removed.

For example, the heterogeneous cell population may trypsinized to create a cell suspension. The suspended heterogeneous cell population may be incubated with magnetic beads-conjugated antibodies and then magnets used to select the desired cells.

In contrast with previous preparations, the RPE cells in the pharmaceutical preparations described herein may survive long term following transplantation. For example, the RPE cells may survive at least about 1, 2, 3, 4, 5, 6, 7, 8, 9, or 10 days. Additionally, the RPE cells may survive at least about 1, 2, 3, 4, 5, 6, 7, 8, 9, or 10 weeks; at least about 1, 2, 3, 4, 5, 6, 7, 8, 9, or 10 months; or at least about 1, 2, 3, 4, 5, 6, 7, 8, 9, or 10 years. Further, the RPE cells may survive throughout the lifespan of the receipt of the transplant. Additionally, at least 10, 20, 30, 40, 50, 60, 70, 80, 90, 95, 96, 97, 98, 99, or 100% of the receipts of RPE cells described herein may show survival of the transplanted RPE cells. Further, the RPE cells described herein may successfully incorporate into the RPE layer in the transplantation receipt, forming a semi-continuous line of cells and retain expression of key RPE molecular markers (e.g., RPE65 and bestrophin). The RPE cells described herein may also attach to the Bruch's membrane, forming a stable RPE layer in the transplantation receipt. Also, the RPE cells described herein are substantially free of ES cells and the transplantation receipts does not show abnormal growth or tumor formation at the transplantation site. The methods described herein resulted in surprisingly ultra-pure isolated populations of RPE cells differentiated from ES cells under sterile conditions in a reduced period of time (compared to manual or chemical selection of RPE cells).

After isolation the cells may remain viable, and may retain the ability to proliferate (whether in vitro or in vivo). The isolated cells may be cultured prior to further use, for example to establish larger populations of cells. Isolated cells may also be used without further proliferation subsequent to isolation. For example, The RPE cells may be cultured under conditions to increase the expression of alpha integrin subunits 1-6 or 9 as compared to uncultured RPE cells or other RPE cell preparations prior to transplantation. The RPE cells described herein may be cultured to elevate the expression level of alpha integrin subunits 1, 2, 3, 4, 5, 6, or 9. The RPE cells described herein may be cultured under conditions that promote the expression of alpha integrin subunits 1-6. For example, the RPE cells may be cultured with integrin-activating agents including but not limited to manganese and the activating monoclonal antibody (mAb) TS2/16. See Afshari, et al. Brain (2010) 133(2): 448-464.

In another embodiment, the RPE cells may be isolated in accordance with Good Manufacturing Practice (GMP). In a further embodiment, the RPE cells may be isolated in accordance with Good Tissue Practice (GTP).

### Selection Criteria for Cells

The method may be used to select and isolate any desired cells. In one preferred embodiment, the desired cells are cells of a particular type, such as RPE cells. The cells may be selected (or excluded from selection) based on any detectable characteristic, including: morphology, pigmentation, expression of a marker gene, level of expression of a particular gene, expression of a detectable marker (such as GFP or another fluorescent protein), autofluorescence (e.g., due to lipofuscin, elastin, or collagen), viability, surroundings (e.g., colony size, morphology, local environment) Cells may also be selected (or excluded from selection) based on any combination of the foregoing types of characteristics.

For example, cells exhibiting characteristics of the desired cell type(s) may be selected for isolation, and optionally cells exhibiting characteristics of undesired cell type(s) may be excluded from selection. Selection may be based on any detectable characteristics, including morphology, pigmentation, detectable markers, and others. For example, pigmentation may be used for selection (or for exclusion from selection) of cell types that may naturally contain brown pigmentation in their cytoplasm: melanocytes, keratinocytes, retinal pigment epithelium (RPE) and iris pigment epithelium (IPE). Further morphological and other characteristics may be used to distinguish among these four cell types before or after isolation. Melanocytes may be distinguished by their non-epithelial morphology, and keratinocytes may be distinguished because they do not produce melanin, but rather only take it up via melanosomes. RPE and IPE cells may be distinguished from melanocytes or keratinocytes by their typical epithelial cobblestone monolayer appearance. RPE and IPE may be further distinguished from one another based on molecular, functional, and morphological characteristics, including: expression of bestrophin, RPE65, CRALBP, and PEDF by RPE; and behavior of RPE in culture (little or no pigment may be seen in proliferating RPE cells, but may be retained in tightly packed epithelial islands or re-expressed in newly established cobblestone monolayer after the cells became quiescent). Additional cell types that may be identified based on pigmentation include neurons of the locus coeruleus (which may contain neuromelanin granules in their cell bodies that cause light scattering, resulting in an azure appearance), dopaminergic neurons including neurons of the substantia nigra (which may contain neuromelanin), pigmented cells of the brainstem, and pigmented cells of the zona reticularis of the adrenal gland. Cells may also be identified based on their composition, e.g., by high numbers of mitochondria (in brown fat). Detection of mitochondria, golgi, and other structures may be facilitated by contact with a vital stain, such as those described herein.

Detectable characteristics of ES cells including but are not limited to presence in a round colony with clear margins; a high nucleus/cytoplasm ratio with prominent nucleoli; rounded cells that lie tightly packed with each other suggesting close cell membrane contact; and expression of at least one markers characteristic of ES cells such as OCT-4, Nanog, TRA-1-60, Stage-specific embryonic antigen-3 (SSEA-3), Stage-specific embryonic antigen-4 (SSEA-4), TRA-1-81, SOX2, and alkaline phosphatase. Further exemplary markers that may be used to detect ES cells include at least one of TRA-2-49/6E, growth and differentiation factor 3 (GDF3), reduced expression 1 (REX1), fibroblast growth factor 4 (FGF4), embryonic cell-specific gene 1 (ESG1), developmental pluripotency-associated 2 (DPPA2), DPPA4, telomerase reverse transcriptase (TERT including hTERT in human cells), SALL4, E-CADHERIN, Cluster designation 30 (CD30), Cripto (TDGF-1), GCTM-2, Genesis, Germ cell nuclear factor, and Stem cell factor (SCF or c-Kit ligand). Additionally, desired cells may be distinguished from other cells by pigmentation. For example, RPE cells are generally darker than other cells. These characteristics may be used for selection of ES cells or for their exclusion from selection. For example, cells may be selected from a population differentiated from ES cells based on presence of detectable characteristics of a desired cell type, and the absence of at least one detectable characteristics of ES cells, thereby reducing the risk that undesired ES cells are among the isolated cells.

Additional detectable characteristics that may be used for selection (or exclusion) of cells include known markers that are characteristic of the desired cell type(s). Any method known in the art for detection of markers may be utilized, including contact with an antibody directly or indirectly coupled to a detectable label. For exemplary methods that may be used, see, e.g., Harlow and Lane, Antibodies: a laboratory manual (CSHL Press, 1988). Table 1 provides illustrative examples of cell types and markers thereof that may be used. Exemplary markers include extracellular proteins and other externally accessible cellular antigens, which may be detected using antibodies or other binding molecules. Additional exemplary markers include intracellular molecules, such as mRNAs, proteins, and small molecules that may be detected in living cells. Exemplary techniques and labels that may be used to detect mRNAs, proteins, and small molecules (such as cAMP and nitrous oxide) in living cells including but are not limited to quenched autoligating FRET probes (Abe & Kool (2006) Proc Natl Acad Sci USA 103(2): 263-8), dual FRET molecular beacons (Santangelo, et al. (2004) Nucleic Acids Res. 32(6): e57), peptide-linked molecular beacons (Nitin, et al. (2004) Nucleic Acids Res. 32(6): e58), linear 2′ O-Methyl RNA probes (Molenaar, et al. (2001) Nucleic Acids Res. 29(17): E89-9), nuclease-resistant molecular beacons (Bratu, et al. (2003) Proc Natl Acad Sci USA 100(23): 13308-13), nanostructured probes (Santangelo, et al. (2006) Ann Biomed Eng. 34(1): 39-50), and further methods described in Tan, et al. (2004) Curr Opin Chem Biol. 8(5): 547-53, Patel (1994) in Drosophila melanogaster: Practical Uses in Cell Biology, Methods in Cell Biology, eds. Goldstein, L. S. B. & Fyrberg, E. (Academic, San Diego) 44: 445-487; Zhang, et al. (2002) Nat Rev Mol Cell Biol. 3(12): 906-18; Cook & Bertozzi, (2002) Bioorg Med Chem. 10(4): 829-40; Kojima, et al. (1998) Anal Chem. 70(13): 2446-53; Adams, et al. (1991) Nature 349(6311): 694-7; Levsky & Singer, (2003) J Cell Sci. 116(Pt 14): 2833-8; Politz & Singer, (1999) Methods 18(3): 281-5; Tyagi & Kramer (1996) Nat Biotechnol. 14(3): 303-8; Hayhurst & Georgiou, (2001) Curr Opin Chem Biol. 5(6): 683-9; Tyagi, et al. (1998) Nat Biotechnol. 16(1): 49-53; Tyagi et al. (2000) Nat Biotechnol. 18(11): 1191-6; and Boulon, et al. (2002) Biochimie. 84(8): 805-13.

Exemplary embodiments include detecting markers using an antibody or other binding molecule coupled to a fluorescent label or other detectable label. Exemplary detectable labels that may be coupled directly or indirectly to an antibody including but are not limited to Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alexa Fluor 647, Alexa Fluor 660, Alexa Fluor 680, Alexa Fluor 700, Alexa Fluor 750 and Alexa Fluor 790, fluoroscein isothiocyanate (FITC), Texas Red, SYBR Green, DyLight Fluors, green fluorescent protein (GFP), TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, TET (6-carboxy-2′,4,7,7′-tetrachlorofluorescein), HEX (6-carboxy-2′,4,4′,5′,7,7′-hexachlorofluorescein), Joe (6-carboxy-4′,5′-dichloro-2′,7′-dimethoxyfluorescein) 5-carboxy-2′,4′,5′,7′-tetrachlorofluorescein, 5-carboxyfluorescein, 5-carboxy rhodamine, Tamra (tetramethylrhodamine), 6-carboxyrhodamine, Rox (carboxy-X-rhodamine), R6G (Rhodamine 6G), phthalocyanines, azomethines, cyanines (e.g. Cy3, Cy3.5, Cy5), xanthines, succinylfluoresceins, N,N-diethyl-4-(5′-azobenzotriazolyl)-phenylamine, aminoacridine, and quantum dots.

The cell population may comprise cells of a species selected from the group consisting of: antelopes, bovines, camels, cats, chevrotains (mouse deer), chimpanzee, cow, deer, dog, giraffes, goat, guinea pig, hamster, hippopotamuses, horse, human, mouse, non-human primate, ovine, peccaries, pig, pronghorn, rabbit, rat, rhesus macaque, rhinoceroses, sheep, tapirs, and ungulates.

For example, a marker may be detected using a primary antibody may be directly coupled (e.g., covalently linked) to a detectable label. A primary antibody may also be indirectly coupled to a detectable label, which may include coupling via a secondary antibody that binds to a primary antibody; coupling through binding partners (such as avidin with biotin, streptavidin with biotin, protein A with Fc, protein G with Fc, protein A/G with Fc, Protein L with Fc, NeutrAvidin with biotin), coupling via an antibody binding to an antigen that may be coupled to the primary antibody, coupling via oligonucleotides (e.g., having complementary sequences). Additional detectable labels and coupling methodologies that may adapted for use with the present methods include those disclosed in U.S. Pat. Nos. 5,281,521; 5,902,727; 5,079,172; 5,665,539; 4,732,847; 6,228,578; 5,132,242; 4,081,245; 4,021,534; 4,481,298; 6,165,798; and 6,117,631. Combinations or chains of the foregoing coupling methodologies may also be used.

Exemplary antibodies that may be used with the present methods include polyclonal, monoclonal, humanized, bispecific, and heteroconjugate antibodies. For example, polyclonal antibodies may be raised against whole cells, purified cell surface antigens, or other preparations as described in Harlow & Lane (1999) Using Antibodies: A Laboratory Manual and antibodies against the desired cell type(s) may be depleted, thereby producing polyclonal antibodies that bind other cell types and allow them to be detected and excluded from selection.

Cells may also be identified for selection or for exclusion from selection using staining methods that may be used while retaining cell viability, such as vital stains. These stains may facilitate identification of living cells or identification of cells containing or associated with structures characteristic of a particular cell type. Exemplary vital stains include eosin (which may be used to stain cytoplasm, collagen muscle fibers, and other eosinophilic structures), propidium iodide (a DNA stain that may differentiate necrotic, apoptotic and viable cells), trypan blue (a diazo dye that is excluded by intact cell membranes and selectively colors dead cells), erythrosine B (excluded from live mammalian cells in culture), Hoechst 33258 and Hoechst 33342 (fluorescent dyes that may label DNA in living cells), and other Hoechst stains. Additional vital stains include 7-nitrobenz-2-oxa-1,3-diazole-phallacidin (fluorescently stains actin cytoskeleton in living cells, see Barak, et al. (1980) Proc Natl Acad Sci USA 77(2): 980-984); liposomes containing N-[7-(4-nitrobenzo-2-oxa-1,3-diazole)]-6-aminocaproyl sphingosine (C6-NBD-ceramide) (stains Golgi apparatus, see Lipsky & Pagano (1985) Science 228(4700): 745-7); PicoGreen (stains mitochondrial DNA, see Ashley, et al. (2005) Exp Cell Res. 303(2): 432-46); phenanthridium (stains nucleic acids, see U.S. Pat. No. 5,437,980) and other vital stains known in the art.

Cell types that may be differentiated from cultured hES cells and isolated using the presently disclosed methods include, but are not limited to, ocular cells such as RPE, RPE-like cells, RPE progenitors, IPE cells, vision-associated neural cells including intemuncial neurons (e.g. “relay” neurons of the inner nuclear layer) and amacrine cells (interneurons that interact at the second synaptic level of the vertically direct pathways consisting of the photoreceptor-bipolar-ganglion cell chain—they are synaptically active in the inner plexiform layer and serve to integrate, modulate and interpose a temporal domain to the visual message presented to the ganglion cell), retinal cells, lens cells, rods, cones, or corneal cells. These cells may be identified based on their morphology, pigmentation, expression of characteristic markers, appearance upon contact with a stain, expression of a fluorescent protein, and other detectable characteristics as known in the art and described above.

The foregoing methods may be used to isolate desired cells while excluding undesired cells. In a preferred embodiment the undesired cell will comprise cells which if administered to a subject could cause an adverse reaction or disease. Specific examples include virally infected (e.g., HIV, hepatitis) cells, other diseased or aberrant cells (e.g., cancerous, precancerous and cancer stem cells), certain immune cells such as T lymphocytes, and the like which if administered to a recipient, could result in infection, disease, or other adverse reaction such as an adverse immune reaction (e.g., GVHD), or result in the proliferation of undesired cells. Other exemplary undesired cells that may be excluded include cell types other than the desired cell types, even though such cells in general may confer little risk of causing adverse reaction or disease. Undesired cell types may be identified by the presence of a detectable characteristic, such as morphology and/or expression of a marker.

In an exemplary embodiment, cells are excluded from selection if they exhibit expression of an undesired cell marker. An undesired cell marker may be specific for one undesired cell type or may indicate several possible undesired cell types. Any marker (or combination of markers) may be used so long as it allows desired and undesired cells to be differentiated. Additionally, an undesired cell marker may exhibit a frequency of “false positive” binding to the desired cell type. Cells that express an undesired cell marker may be treated as undesired cells (e.g., exclusion of that cell from selection and optionally exclusion of cells within a chosen distance of that cell from selection) even though that cell may also exhibit a characteristic indicative of a desired cell type. Combinations of markers may be used to simultaneously indicate a variety of undesired cell types, e.g., the “Lin” markers intended to distinguish between hematopoietic stem cells and other blood cell types (see, e.g., Lagasse, et al. (2000) Nature Medicine 6: 1229-1234). Thus, exemplary embodiments include detection of multiple undesired cell markers and treating any cell that detectably expresses any undesired cell marker as an undesired cell type. Optionally, multiple undesired cell markers may be detected in a manner that does not distinguish among them, for example using multiple antibodies directly or indirectly coupled to the same fluorophore. As one specific example, multiple undesired cell markers may be detected using primary antibodies sharing a common binding moiety (e.g., an Fc of a particular species, coupling to avidin, biotin) and that common binding moiety may be detected using a fluorophore directly or indirectly coupled to a binding molecule that recognizes that common binding moiety (e.g., a secondary antibody specific for that species or another specific binding partner of the common binding moiety).

TABLE 2
Exemplary cell types and markers indicative of those cell types.

| | | |
|---|---|---|
| Marker Name | Cell Type | Significance |

Blood Vessel

| | | |
|---|---|---|
| Fetal liver kinase-1 | Endothelial | Cell-surface receptor protein that |
| (Flk1) | | identifies endothelial cell progenitor; |
| | | marker of cell-cell contacts |
| Smooth muscle cell- | Smooth muscle | Identifies smooth muscle cells in the |
| specific myosin heavy | | wall of blood vessels |
| chain | | |
| Vascular endothelial | Smooth muscle | Identifies smooth muscle cells in the |
| cell cadherin | | wall of blood vessels |

Bone

| | | |
|---|---|---|
| Bone-specific alkaline | Osteoblast | Enzyme expressed in osteoblast; |
| phosphatase (BAP) | | activity indicates bone formation |
| Hydroxyapatite | Osteoblast | Minerlized bone matrix that provides |
| | | structural integrity; marker of bone |
| | | formation |
| Osteocalcin (OC) | Osteoblast | Mineral-binding protein uniquely |
| | | synthesized by osteoblast; marker of |
| | | bone formation |

Bone Marrow and Blood

| | | |
|---|---|---|
| Bone morphogenetic | Mesenchymal stem and | Important for the differentiation of |
| protein receptor | progenitor cells | committed mesenchymal cell types |
| (BMPR) | | from mesenchymal stem and |
| | | progenitor cells; BMPR identifies |
| | | early mesenchymal lineages (stem |
| | | and progenitor cells) |
| B220 | Expressed (typically at high | |
| | levels) on all hematopoietic | |
| | cells. Expression of different | |
| | isoforms is characteristic of | |
| | differentiated subsets of | |
| | hematopoietic cells. B220 | |
| | expression may be used as a | |
| | marker for the B-lymphocyte | |
| | lineage | |
| CD2 | Thymic and peripheral T-cells, | |
| | thymocytes, NK-cells, many | |
| | thymic B-cells, and may be | |
| | expressed also on mature B- | |
| | cells. | |
| CD3 | Thymocytes and T cells | |
| CD4 | thymocyte and T-lymphocytes, | |
| | peripheral blood monocytes, | |
| | tissue macrophages, | |
| | granulocytes | |
| CD5 | thymocytes, T-cells, a small | |
| | subset of mature B-lymphocytes | |
| CD8 | subsets of thymocytes and | |
| | cytotoxic T-cells | |
| CD4 and CD8 | White blood cell (WBC) | Cell-surface protein markers specific |
| | | for mature T lymphocyte (WBC |
| | | subtype) |
| CD34 | Hematopoietic stem cell (HSC), | Cell-surface protein on bone marrow |
| | satellite, endothelial progenitor | cell, indicative of a HSC and |
| | | endothelial progenitor; CD34 also |
| | | identifies muscle satellite, a muscle |
| | | stem cell |
| CD34⁺Sca1⁺ Lin⁻ | Mesencyhmal stem cell (MSC) | Identifies MSCs, which may |
| profile | | differentiate into adipocyte, |
| | | osteocyte, chondrocyte, and myocyte |
| CD38 | Absent on HSC | Cell-surface molecule that identifies |
| | Present on WBC lineages | WBC lineages. Selection of |
| | | CD34⁺/CD38⁻ cells allows for |
| | | purification of HSC populations |
| CD44 | Mesenchymal | A type of cell-adhesion molecule |
| | | used to identify specific types of |
| | | mesenchymal cells |
| c-Kit | HSC, MSC | Cell-surface receptor on BM cell |
| | | types that identifies HSC and MSC; |
| | | binding by fetal calf serum (FCS) |
| | | enhances proliferation of ES cells, |
| | | HSCs, MSCs, and hematopoietic |
| | | progenitor cells |
| Colony-forming unit | HSC, MSC progenitor | CFU assay detects the ability of a |
| (CFU) | | single stem cell or progenitor cell to |
| | | give rise to at least one cell lineages, |
| | | such as red blood cell (RBC) and/or |
| | | white blood cell (WBC) lineages |
| Fibroblast colony- | Bone marrow fibroblast | An individual bone marrow cell that |
| forming unit (CFU-F) | | has given rise to a colony of |
| | | multipotent fibroblastic cells; such |
| | | identified cells are precursors of |
| | | differentiated mesenchymal lineages |
| Gr-1 (Ly6G) | myeloid differentiation antigen | |
| | expressed by myeloid cells in a | |
| | developmentally regulated | |
| | manner in the bone marrow. | |
| | Monocytes only express Gr-1 | |
| | transiently during their | |
| | development in the bone | |
| | marrow. Expressed on bone | |
| | marrow granulocytes and | |
| | peripheral neutrophils. | |
| Hoechst dye | Absent on HSC | Fluorescent dye that binds DNA; |
| | | HSC extrudes the dye and stains |
| | | lightly compared with other cell |
| | | types |
| Leukocyte common | WBC | Cell-surface protein on WBC |
| antigen (CD45) | | progenitor |
| Lineage surface | HSC, MSC | Up to thirteen or fourteen different |
| antigen (Lin) | Differentiated RBC and WBC | cell-surface proteins that are markers |
| | lineages | of mature blood cell lineages; |
| | | detection of Lin-negative cells |
| | | assists in the purification of HSC |
| | | and hematopoietic progenitor |
| | | populations. May include CD13 & |
| | | CD33 for myeloid, CD71 for |
| | | erythroid, CD19 for B cells, CD61 |
| | | for megakaryocytic for humans; and, |
| | | B220 (murine CD45) for B cells, |
| | | Mac-1 (CD11b/CD18) for |
| | | monocytes, Gr-1 for Granulocytes, |
| | | Ter119 for erythroid cells, Il7Ra, |
| | | CD3, CD4, CD5, CD8 for T cells. |
| Mac-1 | WBC; myeloid cells and NK- | Cell-surface protein specific for |
| | cells (granulocytes, monocytes, | mature granulocyte and macrophage |
| | subsets of T-cells and B-cells) | (WBC subtypes) |
| Muc-18 (CD146) | Bone marrow fibroblasts, | Cell-surface protein |
| | endothelial | (immunoglobulin superfamily) |
| | | found on bone marrow fibroblasts, |
| | | which may be important in |
| | | hematopoiesis; a subpopulation of |
| | | Muc-18+ cells are mesenchymal |
| | | precursors |
| NK1.1 | NK cells and some T cells | |
| Stem cell antigen (Sca- | HSC, MSC | Cell-surface protein on bone marrow |
| 1) | | (BM) cell, indicative of HSC and |
| | | MSC Bone Marrow and Blood cont. |
| Stro-1 antigen | Stromal (mesenchymal) | Cell-surface glycoprotein on subsets |
| | precursor cells, hematopoietic | of bone marrow stromal |
| | cells | (mesenchymal) cells; selection of |
| | | Stro-1+ cells assists in isolating |
| | | mesenchymal precursor cells, which |
| | | are multipotent cells that give rise to |
| | | adipocytes, osteocytes, smooth |
| | | myocytes, fibroblasts, chondrocytes, |
| | | and blood cells |
| TER-119 (Ly76) | erythroid lineage cells | |
| Thy-1 | HSC, MSC | Cell-surface protein; negative or low |
| | | detection is suggestive of HSC |

Cartilage

| | | |
|---|---|---|
| Collagen types II and | Chondrocyte | Structural proteins produced |
| IV | | specifically by chondrocyte |
| Keratin | Keratinocyte | Principal protein of skin; identifies |
| | | differentiated keratinocyte |
| Sulfated proteoglycan | Chondrocyte | Molecule found in connective |
| | | tissues; synthesized by chondrocyte |

Fat

| | | |
|---|---|---|
| Adipocyte lipid- | Adipocyte | Lipid-binding protein located |
| binding protein | | specifically in adipocyte |
| (ALBP) | | |
| Fatty acid transporter | Adipocyte | Transport molecule located |
| (FAT) | | specifically in adipocyte |
| Adipocyte lipid- | Adipocyte | Lipid-binding protein located |
| binding protein | | specifically in adipocyte |
| (ALBP) | | |

General

| | | |
|---|---|---|
| Y chromosome | Male cells | Male-specific chromosome used in |
| | | labeling and detecting donor cells in |
| | | female transplant recipients |
| Karyotype | Most cell types | Analysis of chromosome structure |
| | | and number in a cell |

Liver

| | | |
|---|---|---|
| Albumin | Hepatocyte | Principal protein produced by the |
| | | liver; indicates functioning of |
| | | maturing and fully differentiated |
| | | hepatocytes |
| B-1 integrin | Hepatocyte | Cell-adhesion molecule important in |
| | | cell-cell interactions; marker |
| | | expressed during development of |
| | | liver |

Nervous System

| | | |
|---|---|---|
| CD133 | Neural stem cell, HSC | Cell-surface protein that identifies |
| | | neural stem cells, which give rise to |
| | | neurons and glial cells |
| Glial fibrillary acidic | Astrocyte | Protein specifically produced by |
| protein (GFAP) | | astrocyte |
| Microtubule- | Neuron | Dendrite-specific MAP; protein |
| associated protein-2 | | found specifically in dendritic |
| (MAP-2) | | branching of neuron |
| Myelin basic protein | Oligodendrocyte | Protein produced by mature |
| (MPB) | | oligodendrocytes; located in the |
| | | myelin sheath surrounding neuronal |
| | | structures |
| Nestin | Neural progenitor | Intermediate filament structural |
| | | protein expressed in primitive neural |
| | | tissue |
| Neural tubulin | Neuron | Important structural protein for |
| | | neuron; identifies differentiated |
| | | neuron |
| Neurofilament (NF) | Neuron | Important structural protein for |
| | | neuron; identifies differentiated |
| | | neuron |
| Neurosphere | Embryoid body (EB), ES | Cluster of primitive neural cells in |
| | | culture of differentiating ES cells; |
| | | indicates presence of early neurons |
| | | and glia |
| Noggin | Neuron | A neuron-specific gene expressed |
| | | during the development of neurons |
| O4 | Oligodendrocyte | Cell-surface marker on immature, |
| | | developing oligodendrocyte |
| O1 | Oligodendrocyte | Cell-surface marker that |
| | | characterizes mature |
| | | oligodendrocyte |
| Synaptophysin | Neuron | Neuronal protein located in |
| | | synapses; indicates connections |
| | | between neurons |
| Tau | Neuron | Type of MAP; helps maintain |
| | | structure of the axon |

Pancreas

| | | |
|---|---|---|
| Cytokeratin 19 (CK19) | Pancreatic epithelium | CK19 identifies specific pancreatic |
| | | epithelial cells that are progenitors |
| | | for islet cells and ductal cells |
| Glucagon | Pancreatic islet | Expressed by alpha-islet cell of |
| | | pancreas |
| Insulin | Pancreatic islet | Expressed by beta-islet cell of |
| | | pancreas Pancreas |
| Insulin-promoting | Pancreatic islet | Transcription factor expressed by |
| factor-1 (PDX-1) | | beta-islet cell of pancreas |
| Nestin | Pancreatic progenitor | Structural filament protein indicative |
| | | of progenitor cell lines including |
| | | pancreatic |
| Pancreatic polypeptide | Pancreatic islet | Expressed by gamma-islet cell of |
| | | pancreas |
| Somatostatin | Pancreatic islet | Expressed by delta-islet cell of |
| | | pancreas |

Pluripotent Stem Cells

| | | |
|---|---|---|
| Alkaline phosphatase | Embryonic stem (ES), | Elevated expression of this enzyme |
| | embryonal carcinoma (EC) | is associated with undifferentiated |
| | | pluripotent stem cell (PSC) |
| Alpha-fetoprotein | Endoderm | Protein expressed during |
| (AFP) | | development of primitive endoderm; |
| | | reflects endodermal differentiation |
| | | Pluripotent Stem Cells |
| Bone morphogenetic | Mesoderm | Growth and differentiation factor |
| protein-4 | | expressed during early mesoderm |
| | | formation and differentiation |
| Brachyury | Mesoderm | Transcription factor important in the |
| | | earliest phases of mesoderm |
| | | formation and differentiation; used |
| | | as the earliest indicator of mesoderm |
| | | formation |
| Cluster designation 30 | ES, EC | Surface receptor molecule found |
| (CD30) | | specifically on PSC |
| Cripto (TDGF-1) | ES, cardiomyocyte | Gene for growth factor expressed by |
| | | ES cells, primitive ectoderm, and |
| | | developing cardiomyocyte |
| GATA-4 gene | Endoderm | Expression increases as ES |
| | | differentiates into endoderm |
| GCTM-2 | ES, EC | Antibody to a specific extracellular- |
| | | matrix molecule that is synthesized |
| | | by undifferentiated PSCs |
| Genesis | ES, EC | Transcription factor uniquely |
| | | expressed by ES cells either in or |
| | | during the undifferentiated state of |
| | | PSCs |
| Germ cell nuclear | ES, EC | Transcription factor expressed by |
| factor | | PSCs |
| Hepatocyte nuclear | Endoderm | Transcription factor expressed early |
| factor-4 (HNF-4) | | in endoderm formation |
| Nestin | Ectoderm, neural and pancreatic | Intermediate filaments within cells; |
| | progenitor | characteristic of primitive |
| | | neuroectoderm formation |
| Neuronal cell-adhesion | Ectoderm | Cell-surface molecule that promotes |
| molecule (N-CAM) | | cell-cell interaction; indicates |
| | | primitive neuroectoderm formation |
| OCT4/POU5F1 | ES, EC | Transcription factor unique to PSCs; |
| | | essential for establishment and |
| | | maintenance of undifferentiated |
| | | PSCs |
| Pax6 | Ectoderm | Transcription factor expressed as ES |
| | | cell differentiates into |
| | | neuroepithelium |
| Stage-specific | ES, EC | Glycoprotein specifically expressed |
| embryonic antigen-3 | | in early embryonic development and |
| (SSEA-3) | | by undifferentiated PSCs |
| Stage-specific | ES, EC | Glycoprotein specifically expressed |
| embryonic antigen-4 | | in early embryonic development and |
| (SSEA-4) | | by undifferentiated PSCs |
| Stem cell factor (SCF | ES, EC, HSC, MSC | Membrane protein that enhances |
| or c-Kit ligand) | | proliferation of ES and EC cells, |
| | | hematopoietic stem cell (HSCs), and |
| | | mesenchymal stem cells (MSCs); |
| | | binds the receptor c-Kit |
| Telomerase | ES, EC | An enzyme uniquely associated with |
| | | immortal cell lines; useful for |
| | | identifying undifferentiated PSCs |
| TRA-1-60 | ES, EC | Antibody to a specific extracellular |
| | | matrix molecule is synthesized by |
| | | undifferentiated PSCs |
| TRA-1-81 | ES, EC | Antibody to a specific extracellular |
| | | matrix molecule normally |
| | | synthesized by undifferentiated |
| | | PSCs |
| Vimentin | Ectoderm, neural and pancreatic | Intermediate filaments within cells; |
| | progenitor | characteristic of primitive |
| | | neuroectoderm formation |

Skeletal Muscle/Cardiac/Smooth Muscle

| | | |
|---|---|---|
| MyoD and Pax7 | Myoblast, myocyte | Transcription factors that direct |
| | | differentiation of myoblasts into |
| | | mature myocytes |
| Myogenin and MR4 | Skeletal myocyte | Secondary transcription factors |
| | | required for differentiation of |
| | | myoblasts from muscle stem cells |
| Myosin heavy chain | Cardiomyocyte | A component of structural and |
| | | contractile protein found in |
| | | cardiomyocyte |
| Myosin light chain | Skeletal myocyte | A component of structural and |
| | | contractile protein found in skeletal |
| | | myocyte |

Ocular Cells

| | |
|---|---|
| MP20; connexin 46 | Lens |
| B7-2 (CD86) | Iris Pigment Epithelium |
| Prox1; Lim1; | Horizontal interneurons |
| calbindin; Nfasc; | |
| 6330514A18Rik | |
| Chx10; | Bipolar cells |
| 2300002D11Rik; | |
| 6330514A18Rik; | |
| Car8; Car10; Cntn4; | |
| Lhx3; Nfasc; Og9x; | |
| Scgn; Trpm1; Pcp2; | |
| Grm6 | |
| Calbindin; HPC-1 | Amacrine Cells |
| (syntaxin 1A); | |
| 6330514A18Rik; | |
| Car10; Cntn4; Nfasc | |
| Rhodopsin; recoverin; | Rods |
| peripherin-2; rod | |
| arrestin; | |
| 6330514A18Rik; | |
| Nfasc | |
| Rhodopsin; 7G6; X- | Cones |
| arrestin; calbindin; | |
| recoverin; peripherin- | |
| 2; photopsins; | |
| 6330514A18Rik; | |
| Nfasc | |
| Keratin 3; Keratin 12 | Corneal epithelium |
| Cytokeratin 8; | Corneal endothelium |
| Cytokeratin 18 | |

The present invention will now be more fully described with reference to the following examples, which are illustrative only and should not be considered as limiting the invention described above.

### EXAMPLES

### Example 1

### Protocol for Laser Microdissection of Living In Vitro Cells

### Introduction

Laser capture microdissection (LCM) is a proven technique for the isolation of pure cell populations for downstream molecular analysis. The combined use of UV laser cutting with LCM using an infrared (IR) laser permits rapid and precise isolation of larger numbers of cells while maintaining cellular and nucleic acid integrity necessary for downstream analysis. In this application note, it is shown that these established techniques can also be used for the isolation of living cells, avoiding other more laborious methods of cell selection and enabling a wide range of research applications. This example describes a protocol for the isolation of living adherent cells and the subsequent recultivation of homogeneous subpopulations.

### Methods

### Specimen Preparation

PEN membrane slide may be hourly rinsed with 100% ethanol and air-dry prior to use and keep in a sterile environment (e.g., slide should be completely dry prior to use.) Adherent cells may be trypsinized from a growth vessel (e.g., plate, flask) using a standard protocol. The tyrpsin may be deactivated with medium using a standard protocol. About 1-2 mL of trypsinized cells may be resuspended in about 10 mL of fresh medium. A metal frame membrane slide with chamber may be placed facing up into a sterile Petri dish. About 1 mL of the cell suspension may be transferred into the chamber of the frame membrane slide. If necessary, the slide may be rocked in the Petri dish to completely cover the chamber area with medium. The lid may be placed on the Petri dish and incubated using appropriate culturing conditions for the cells until desired cell confluency is achieved (e.g., replace with fresh medium as needed.

### Laser Microdissection Slide Preparation

The instrument and work area should be thoroughly cleaned, including pipettors, pipette tip box, with 100% ethanol and RNase AWAY® or RNaseZap®. A cover glass may be rinsed with 100% ethanol and air-dry prior to use and in a sterile environment (the cover glass should be completely dry prior to use.) When cells have reached the desired confluency, the medium may be removed from the chamber using a sterile pipette tip. About 950-1,000 μL of fresh medium may be added to the chamber. A cover glass may be placed over the chamber side of the frame slide to create a mini-environment for the cell culture, enabling extended survival and reducing the possibility of the cells drying out. Care should be taken to reduce the amount of air bubbles formed when applying the cover glass. A Kimwipe may be used to carefully blot any excess medium that has seeped outside the cover glass. The slide may be transported in the Petri dish to a Veritas® or Arcturus^{XT}® system.

The slide may be removed from the Petri dish and a Kimwipe soaked in 100% ethanol may be used to clean the flat side of the frame slide. The slide should be dried completely. Care should be taken not to rupture the membrane. The frame slide should be inserted with the chamber and cover glass facing down (flat side up) onto the Veritas® or Arcturus^{XT}® instrument and proceed to laser microdissection.

### Laser Microdissection Protocol

CapSure® Macro LCM Caps may be used. Cut and capture may be performed using light microscopy at 10× or 20×. It is recommended to identified the desired cell, capture the area, and then cut with the laser. The visualizer should be turned off (Veritas™ system) and the diffuser should be removed (Arcturus^{XT}™ system).

The cells of interest to be captured may be identified. The Cut Line feature may be used to draw around cells. The Single Point Capture feature may be used to apply LCM spots that will fuse LCM membrane to PEN membrane. It is preferred to apply an adequate number of LCM spots for the given region.

A CapSure® Macro LCM Cap may be placed onto the area of the slide containing cells of interest. LCM laser may be located and fired at a test LCM shot. If necessary, the laser settings may be adjusted. It is further recommended that the user confirm that the LCM film makes contact with PEN film. (The LCM spot will be dark).

The UV cutting laser may be located. The LCM laser should be activated first and then the UV cutting laser. The Macro LCM Cap may be used to a QC station and the presence of cells on the LCM Cap may be confirmed. The cap may then be moved to an offload station.

TABLE 3
Exemplary Cutting (UV) Laser Settings

| | |
|---|---|
| UV laser power | Veritas system: 20-25 Arcturus^{XT} ® system |
| | (all ND filters out) |
| UV spacing | Veritas system = 5000 μm Arcturus^{XT} ® |
| | system = 5000 μm |
| Tab size/length | Veritas system = 1 Arcturus^{XT} ® system = 0 |
| Automatic LCM spots | Veritas system = 0 Arcturus^{XT} ® system = 0 |
| UV cut speed | Veritas system = N/A Arcturus^{XT} ® |
| | system = 525 |

TABLE 4
Exemplary Capture (IR) Laser Settings

| | |
|---|---|
| IR laser power | Veritas system = 80 Arcturus^{XT} ® system = 65 mW |
| Pulse/Duration | Veritas = system 4000 ms Arcturus^{XT} ® system = 22 ms |
| LCM spot | Veritas = system 40% Arcturus^{XT} ® system = 60% |
| overlap | |

These settings may be used for protocol validation and should be used as a guideline for the microdissection of live cells. Optimization of settings may be required, depending on the individual cell preparation.

### Reculturing Captured Live Cells

The Macro LCM Cap may be removed from the offload station and inverted. The cap with isolated cells may be placed facing up into a clean Petri dish. About 50 μL of Hanks' solution may be pipetted onto the Macro LCM cap film surface. The solution may be pipetted up and down 2-3 times, and the solution disposed. About 50 μL of trypsin-EDTA may be pipetted directly onto the captured cells on the cap and incubated for at least about 5 minutes at room temperature. The Petri dish may be covered with a lid during this incubation. After incubation, trypsin-EDTA may be pipetted up and down several times to ensure a single-cell suspension, then transferred the cell suspension into a well of a sterile chamber slide (or alternate desired growth vessel) containing about 1-2 mL of appropriate cell medium. The chamber slide may be incubated in the incubator under appropriate conditions. Cell growth may be monitored using standard culture techniques, changing medium as needed. The recultured cells may be used as desired for further experiments.

Protocol adapted from “Applied Biosystems® Arcturus^{XT}™ Microdissection Systems: Optimized Protocol for Laser Microdissection of Living In Vitro Cells.” by Applied Biosystems® (2010).

### Example 2

### ES Cell Differentiation to Produce RPE Cells

Human RPE cells were produced by differentiation of human ES cells essentially as described in U.S. Pat. No. 7,795,025. In brief, hES cell cultures were maintained and expanded on mouse embryo fibroblast (MEF) feeder cells, then trypsinized and cultured on low adherent plates (Costar) until embryoid bodies formed. The embryoid bodies were cultured until regions containing pigmented cells having epithelial morphology were formed therein. The embryoid bodies were then digested with enzymes (trypsin, and/or collagenase, and/or dispase), and pigmented cells were selectively picked, plated, and cultured. After about two weeks in culture at low density, the cultured cells lost their pigmentation, but after another two to three weeks in culture regions of pigmented cells having a cobblestone, epithelial-like morphology again appeared. This pigmentation behavior—temporary loss from cells in proliferating cultures, and restoration in quiescent (non-proliferating) cultures over time—is a known characteristic of RPE cells and provided initial confirmation that the culture contained RPE cells. Further confirmation was obtained by detecting expression of molecular markers characteristic of RPE cells. The resulting cultures of RPE cells were passaged and expanded for further use.

### Example 3

### Isolation of Viable RPE Cells Using Laser Microdissection

Culture containing RPE cells differentiated from human ES cells were produced as described in the preceding example. Laser microdissection was then used to isolate islands of pigmented epithelial cells for further culture. ES-derived RPE cells were grown in multiwell culture plates and maintained as quiescent cultures until pigmented epithelial islands were perceptible (e.g., at least about 7 days). The multiwell plate was then placed on a microscope fitted with the STILETTO® laser system (Hamilton Thorne Ltd., Beverly, Mass.) Islands of pigmented epithelial cells were then visualized, and the provided control software was used to manually draw a target zone circumscribing and immediately outside of each pigmented island. Cells in the target zone were then ablated by laser pulses which were caused to strike the target zone by computer-controlled movement of the microscope stage. After ablation of the target zone, each island of pigmented cells was then physically removed using a micromanipulator and further cultured.

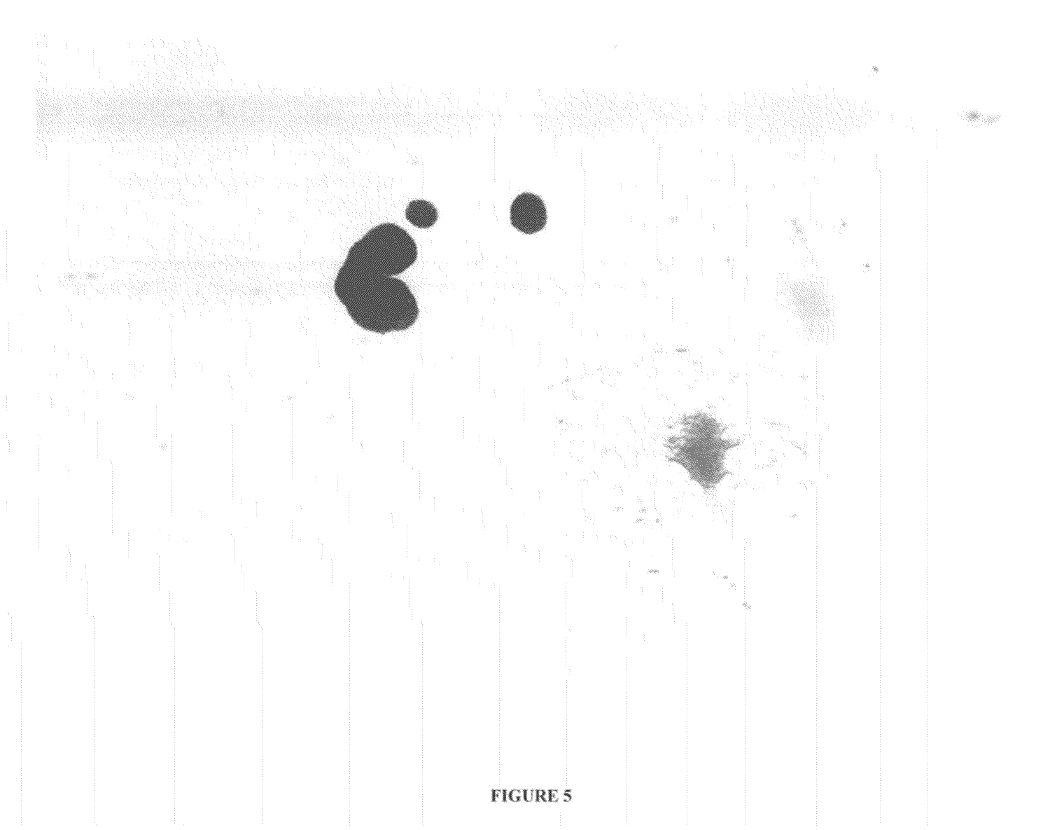

The laser-isolated RPE cells were grown in culture to confluence and then maintained as quiescent cultures until pigmented epithelial islands were established. Compared to control populations of manually selected pigmented epithelial cells, the cultures of laser-isolated cells contained non-pigmented or non-epithelial cells as a proportion of the total number of cells at the similar levels as manually selected clusters. See FIG. 5.

The inventors surprisingly discovered that the laser isolation method was substantially faster than manual colony picking methods (e.g., hours versus days). This is a substantial improvement over manual colony picking methods because it allows for a large number of cells (>10⁶) to be isolated at near purity in a shorter time. This more rapid and effective method of isolating RPE cells from an ES cell population minimizes the time window required to isolate RPE cells and maximizes the time window the isolated RPE cells are available for therapeutic use (e.g., 48 hours). Further, the laser microdissection method allowed the inventors to more rapidly scale up and greatly increase the number of RPE cells in a shorter period of time with less lot-to-lot variance.

### Example 4

### Comparison of Laser-Isolation Methodologies

As in the preceding example, ES-derived RPE cells were grown in multiwell culture plates and maintained as quiescent cultures until pigmented epithelial islands (surrounded by non-pigmented or non-epithelial cells) were established. The RPE cells were then laser-isolated as in the preceding example, except that the target zones were drawn inside the pigmented epithelial islands (instead of immediately outside of the pigmented epithelial islands). The target zones were inside of the boundary of each pigmented epithelial island within 1-2 microns. See, e.g., FIG. 2. The pigmented cells were then isolated and cultured as in the preceding example.

Compared to the laser-isolated cells of the preceding example, the cultures of laser-isolated cells contained a smaller proportion of non-pigmented or non-epithelial cells. Thus, laser isolation by cutting within the boundaries of the pigmented epithelial islands produced higher-purity RPE cultures than laser isolation by cutting just outside of the boundaries of the pigmented epithelial islands.

### Example 5

### Multiple Rounds of Purification to Produce Higher Purity RPE Cultures

RPE cells are produced from hES cells and then laser-purified as described in the preceding examples (with laser cutting either immediately surrounding or within pigmented epithelial islands). The laser-purified RPE cells are cultured until pigmented epithelial islands appear. A second-round of laser-isolation is then carried out, resulting in a twice-isolated population of RPE cells. Cultures arising from twice-isolated cells contain an even greater proportion of pigmented epithelial cells. The twice-isolated cells may again be cultured until pigmented epithelial islands appear, and yet again laser isolated to produce a three times-isolated population of pigmented epithelial cells. Further rounds of laser isolation may be performed until a desired degree of purity is achieved.

### Example 6

### Laser Isolation of Other Eye Cell Types

A population of cells is differentiated from embryonic stem cells using the method described in Example 1. A desired eye cell type (such as ocular cells including RPE, RPE-like cells, RPE progenitors, IPE cells, vision-associated neural cells, internuncial neurons, amacrine cells, retinal cells, lens cells, rods, cones, or corneal cells) are identified based on morphology, pigmentation, expression of characteristic markers, appearance upon contact with a stain, or other detectable characteristics. An antibody to a marker characteristic of the desired cell type (coupled directly or indirectly to a detectable label) may be used to facilitate detection. Cells of the desired type are then isolated for further culture. An initial isolation is performed using laser isolation or other means (e.g., mechanical picking). The isolated cells are then cultured. The desired cell type may then undergo at least one rounds of laser isolation, thereby producing a more pure culture of the desired cell type. The isolated cells may then be used for cell-based therapy in a human or non-human animal.

While the invention has been described by way of examples and preferred embodiments, it is understood that the words which have been used herein are words of description, rather than words of limitation. Changes may be made, within the purview of the appended claims, without departing from the scope and spirit of the invention in its broader aspects. Although the invention has been described herein with reference to particular means, materials, and embodiments, it is understood that the invention is not limited to the particulars disclosed. The invention extends to all equivalent structures, means, and uses which are within the scope of the appended claims.

Although the invention has been described in some detail by way of illustration and example for purposes of clarity of understanding, it was obvious that certain changes and modifications may be practiced within the scope of the appended claims. Modifications of the above-described modes for carrying out the invention that are obvious to persons of skill in cell biology, molecular biology, and/or related fields are intended to be within the scope of the following claims.

All publications (e.g., Non-Patent Literature), patents, patent application publications, and patent applications mentioned in this specification are indicative of the level of skill of those skilled in the art to which this invention pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

1-66. (canceled)

67. A method for isolating a viable cell from a heterogeneous population of cells comprising
(a) providing a planar carrier on which said population of cells containing said at least one viable cell is situated,
(b) placing said culture dish in a microscope coupled to a laser microdissection system,
(a) selecting said viable cell,
(b) excising said viable cell,
(c) separating said viable cell from the planar carrier, and
(d) collecting said viable cell.

68-78. (canceled)

79. The method of claim 67, wherein said viable cell is produced by culturing pigmented epithelial cells obtained from differentiated embryonic stem cells.

80. The method of claim 67, wherein said viable cell is an RPE cell selected based on pigmentation.

81. The method of claim 67, wherein said viable cell is an RPE cell selected based on at least one detectable characteristic of RPE cells.

82. The method of claim 81, wherein said detectable characteristic of RPE cells includes at least one of presence of brown pigmentation accumulated within the cytoplasm, a cobblestone, epithelial-like morphology, or expression of at least one RPE cell markers.

83. The method of claim 82, wherein said RPE cell marker is selected from the group consisting of bestrophin, RPE65, CRALBP, and PEDF.

84. The method of claim 83, wherein said marker is detected by a method selected from the group consisting of binding to an antibody directly or indirectly coupled to a detectable label; incubation with magnetic beads-conjugated antibodies; detecting the expression of a fluorescent protein; detecting an intracellular mRNA, detecting an intracellular protein; and detecting an intracellular small molecule.

85. (canceled)

86. The method of claim 67, wherein excising of step (d) comprises removing the selected cells from the planar carrier using micromanipulation or laser catapulting.

87. (canceled)

88. The method of claim 67, wherein said collected viable cells essentially comprise no undifferentiated cells.

89-95. (canceled)

96. The method of claim 67, wherein said laser light is ultraviolet light.

97. The method of claim 67, wherein said laser light is provided as pulses having a duration between about 100 μs and about 3000 μs.

98. The method of claim 67, wherein said laser light is produced from a laser selected from the group consisting of argon ion lasers, diode lasers, dye lasers, excimer lasers, fiber lasers, free electron lasers, krypton ion lasers, Nd: YAG lasers, Nd: YVO₄ lasers, and solid-state bulk lasers.

99. (canceled)

100. (canceled)

101. (canceled)

102. A method for isolating a RPE cell from a population of cells comprising
(a) providing a planar carrier on which said population of cells is situated,
(b) placing said planar carrier in a microscope coupled to a laser microdissection system,
(c) selecting said at least one RPE cell,
(d) excising said cell from undesired cells or other materials in target areas adjacent to the selected cells using laser light, thereby severing the connections between the selected cells and adjacent cells or other materials, and
(e) collecting said RPE cell.

103. The method of claim 102, wherein said RPE cell is selected from the group consisting of iris pigment epithelium cells, vision-associated neural cells, lens cells, rod cells, cone cells, or corneal cells.

104. The method of claim 102, wherein said population of cells is a heterogeneous population.

105. The method of claim 102, wherein said RPE cell is differentiated from one or more pluripotent cells.

106-138. (canceled)

139. A method of isolating a viable RPE cell from a heterogeneous population of cells comprising
(a) providing a planar carrier on which a cell population comprising at least one viable desired cell is situated;
(b) selecting at least one desired cell to be isolated;
(c) excising said at least one cell from undesired cells or other materials in target areas adjacent to the selected cells using laser light, thereby severing the connections between the selected cells and adjacent cells or other materials; and
(d) separating the at least one selected cell from the planar carrier, thereby isolating the selected cells, wherein the isolated cells comprise viable desired cells, wherein said desired cells are of a desired cell type selected from the group consisting of iris pigment epithelium cells, vision-associated neural cells, lens cells, rod cells, cone cells, or corneal cells.

140. The method of claim 139, wherein said RPE cell is differentiated from one or more pluripotent cells.

141. (canceled)

142. (canceled)

143. The method of claim 139, wherein said selected cell exhibits at least one detectable characteristics of RPE cells.

144. The method of claim 143, wherein said detectable characteristics of RPE cells includes morphology or expression of at least one RPE cell markers.

145-180. (canceled)
